(19) United States
(12) Patent Application Publication
(10) Pub. No.: US 2006/0271061 A1
(43) Pub. Date: Nov. 30, 2006

(54) Deformable tools and implants

(75) Inventors: Modechay Beyar, Caesarea (IL); Ronen Shavit, Tel-Aviv (IL); Oz Vachtenberg, Tel-Aviv (IL); Oren Globerman, Kfar-Shmaryahu (IL); Uri Shasha, Ramat-Gan (IL)

Correspondence Address: WOLF, BLOCK, SCHORR & SOLIS-COHEN LLP, 250 PARK AVENUE, NEW YORK, NY, 10177, US

(73) Assignee: Disc-o-tech, Ltd., Herzlia Pituach (IL)

(21) Appl. No.: 10/561,124

(22) PCT Filed: Jun. 17, 2004
(86) PCT No.: PCT/IL04/00527

§ 371 Date: Jun. 15, 2006

Related U.S. Application Data
(60) Provisional application No. 60/478,841, filed on Jun. 17, 2003.

(51) Int. Cl. A61B 17/68; A61F 2/44

(52) U.S. Cl. 606/105; 623/17.11

(57) ABSTRACT

A medical grade deformer, comprising: an axial member; and a pliable tube mounted on said axial member and adapted to be deformed from a first, narrower diameter, configuration to a second, greater diameter, configuration.

### RELATED APPLICATIONS

The present application claims the benefit under 35 USC 119(e) of U.S. Provisional Applications for patent Ser. No. 60/478,841 filed on Jun. 17, 2003, Ser. No. 60/529,612 filed on Dec. 16, 2003, Ser. No. 60/534,377 filed Jan. 6, 2004 and Ser. No. 60/554,558 Mar. 18, 2004, the disclosures of which are incorporated herein by reference.

The present application is a continuation-in-part of U.S. Ser. No. 09/890,172 and U.S. Ser. No. 09/890,318, the disclosures of which are incorporated herein by reference.

The present application claims priority from Israel application IL 160987, filed 21 Mar. 2004, the disclosure of which is incorporated wherein by reference.

### FIELD OF THE INVENTION

The present invention is related to tools and implants which are deformed during use, for example for orthopedic use.

### BACKGROUND

Spinal compression fractures are painful and disfiguring fractures in which a spinal vertebra axially compresses. This causes distortion and shortening of the spine and also pain, like other fractures. A conventional treatment is bed rest, which heals the fracture but does not reset the bone in its original configuration.

US patent application publication 2002/0156482A1 Scribner et al., the disclosure of which is incorporated herein by reference, describes a procedure by which a balloon is inserted into a spinal vertebra and then expanded, in an attempt to reset the vertebra to its original configuration. Thereafter, bone filler may be injected into a void created by the balloon, to fixate the bone.

PCT publication WO 00/44319, the disclosure of which is incorporated herein by reference, suggests a device which can be mechanically expanded to support two vertebrae or to be inserted inside a bone to support a fracture.

### SUMMARY OF THE INVENTION

An aspect of some embodiments of the invention relates to a method of treating a vertebra in which a plastic (e.g., polymeric, non-metallic) deformer is inserted into the vertebra and deformed to have an increased dimension in the axis of the spine, without use of inflation via fluid injection or absorbing. In an exemplary embodiment of the invention, the deformer is deformer by applying force (e.g., a compression force) thereto. In another embodiment, the deformer is released to retain an original configuration thereof. In an exemplary embodiment of the invention, a kit including a suitably sized deformer and a spinal delivery system, is provided. A potential advantage of some plastic materials is their biocompatibility and a wide range of mechanical properties, such as elongation. In an exemplary embodiment of the invention, different axially positioned sections of the deformer support each other in a direction perpendicular to the direction of force applied by the vertebra. Optionally, the sections are axially dense in that each section leans, over a substantial part of its length, on a next section. Optionally, the sections are contiguous (e.g., a solid deformer). In an alternative embodiment, one section is deformed on (and/or confirms to) a neighboring section, during deployment.

An aspect of some embodiments of the invention relates to a pliable medical element (hereafter a “deformer”) configured to deform from filling a narrow diameter volume to filling or defining a larger diameter volume. In an exemplary embodiment of the invention, the deformer comprises a slotted tube, formed of a polymer and/or fabric material, such that when the tube is axially compressed, a plurality of leaves extend to the sides of the tube and fill the volume surrounding the tube. Optionally, a locking element, for example an inner bolt or wire is used to maintain the tube in its deformed configuration. In another embodiment, the deformer is formed of an unslotted tube.

In an exemplary embodiment of the invention, the material used is pliable enough to provide a deformer with some degree of conforming to a body geometry, for example cortical plates of a vertebra, while still being able to apply a force against an external resisting body (such as the plates). This is in contrast to cement, which simply flows to where there is least resistance, even through cracks and out of the vertebra This is also in contrast to a balloon, which, being filled with a fluid, tends to equalizes pressures on different parts thereof, thus force is applied in a direction of least resistance.

In an exemplary embodiment of the invention, a deformer is used to apply a desired degree of deformation and displacement, in contrast to a balloon that can apply a force, but not determine a displacement. In some embodiments of the invention, the deformation extent, displacement and/or deformation force are controllable and/or pre-configured.

One potential disadvantage of some implementations of a balloon is that expanding a balloon near a vertebral wall may cause application of force on the wall and fracturing thereof. A deformer, in accordance with exemplary embodiments of the invention, can be deformed with a small safety margin (e.g., distance), for example, 1 mm, 2, mm, 3 mm, 4 mm or less, from the vertebral cortical bone which is in danger of damage from application of force.

In an exemplary embodiment of the invention, a deformer is used for a spinal application, for example as a tool for expanding a compressed vertebra and/or as an implant for spacing between or inside a vertebra. Optionally, the force which can be applied and/or withstood by a deformer in a deformed state is greater than 10 Kg, 20 Kg, 50 Kg, 70 Kg, 100 Kg or and smaller, intermediate or greater value.

In an exemplary embodiment of the invention, a deformer is deformed using the following process. A distal end of the deformer is (optionally) held in place while a proximal end of the deformer is pushed or pulled axially towards the distal end. An overtube surrounding the deformer is optionally provided to control the deformation process. Optionally, the overtube is retracted in conjunction with the pushing (or pulling), to provide serial deformation of the deformer. One potential advantage of the optional maintaining the position of the distal end of the deformer, is that the deformer can thus deform in place without retracting from (and/or otherwise moving relative to) tissue.

The deformer can have various shapes once deformed, for example, the deformer can be cylindrical, cone line (with a truncated tip) or lordotic (a truncated 4 sided pyramid). In some embodiments of the invention, the deformer has rotational symmetry around the axis of the tube. In other embodiments, for example for fitting in asymmetric gaps or areas where cortical bones should not be parallel, an asymmetric design is used. For example, the deformer may be concave on one side. In another example, the axis of the deformer bends when the deformer is deforming. In another example, the cross-section of the deformer is non-uniform and/or rotationally asymmetric. Optionally, the pliability and/or other mechanical properties, such as flexural modulus, tensile strength and/or elongation of the deformer vary along its length and/or at different angles thereof.

In an exemplary embodiment of the invention, neighboring leaves of the deformer support each other when the deformer is deformed. Optionally, the leaves at one or both ends of the deformer are shorter, so that they can make do with support from only one side of leaves. Optionally, alternatively or additionally, the end leaves are made less pliable. Optionally, alternatively or additionally, the end leaves are designed to extend axially and not only radially as in some other designs.

Optionally, the leaves of a deformer substantially fill the space outside the deformer, in the volume defined thereby, for example, 30%, 40%, 50%, 60%, 70%, 80%, 90% or more of the volume excluding a volume of an inner rod. The rest of the volume is optionally filled with fluid (from the body), bone marrow, cement, filler, or vertebral bone. It should be noted that the expansion of the vertebra may cause gaps to appear in the bone.

While a slotted tube is described, in other embodiments, other base forms are used for the deformer. For example, an unslotted tube may be used. In another example, slots are not through the entire thickness of the tube.

In an exemplary embodiment of the invention, the deformer is used as an implant. Optionally, the implant is delivered on a delivery tube and then released. In an exemplary embodiment of the invention, the deformer implant includes two end plates that are interconnected by a bar or wire. The end plates, optionally made of metal, are optionally used to apply compressive force in an axial direction against the ends of the pliable ends of the deformer. This type of implant may be used, for example, in a spinal vertebra, or for supporting fallen end plates in long bones.

Optionally, the implant includes one or more radio-opaque markers and/or the end plates act as such markers.

In an exemplary embodiment of the invention, the deformer is used as a void creating and/or tissue moving tool. In an exemplary embodiment of the invention, the deformer is mounted on a rod, one end of which rod is fixed to a first end of the deformer and another end of which rod is coupled to an element that pushes a second end of the deformer towards the first end. One or more radio-opaque markers may be provided.

In an exemplary embodiment of the invention, the deformer is used as a tissue engaging element. In one example, the deformer is used to hold an implant inside a medullar channel of a bone, for example, for holding a prosthesis, for holding an intra-medullar nail or for holding a femoral head screw. In another example, two deformers are used, each one holding a different tissue section and optionally interconnected by a bar, wire or hinge.

In an exemplary embodiment of the invention, while being deformed, the volume taken up by the deformer is reduced, for example, due to compression of the deformer material (e.g., made of a porous, fabric or compressible material), or due to compression of voids formed in the material. Optionally, alternatively or additionally, the deformer is compressed into an inner channel thereof (e.g., a lumen of a tube deformer), so that the total external volume defined by the deformer is reduced, for example, by 5%, 10%, 20% or more.

Optionally, the deformer has a composite structure. In one embodiment of the invention, one or more threads, for example of metal or Kevlar are embedded in the deformer, for example, to increase tensile strength and/or to modify mechanical properties of the deformer. Optionally, alternatively or additionally, the deformer is composed of axial segments that are welded or otherwise attached, each segment having different properties. For example, 2, 3, 4, 5 or more different material properties or different sections may be provided in a deformer.

Optionally, the deformer is configured to elute a material with biochemical properties, for example, a bone growth enhancing material. Optionally, the deformer is coated with such a material or a delivery vehicle for the material. Alternatively or additionally, for example, the deformer is impregnated with the material and/or includes one or more void filled with such material, so that when deformed, the material is eluted. Optionally, the eluted material is a cement or a cement hardener.

An aspect of some embodiments of the invention relates to a method of treating a spinal compression fracture or a fallen bone plate in a long bone, using a deformer. In an exemplary embodiment of the invention, a deformer is guided into a vertebra, for example using a guide wire and then deformed so that the vertebra is axially expanded. Possibly, an old fracture can be re-broken using this method. Then, the deformer is removed and cement, bone substitute, or bone slurry is injected and used to set the vertebra. Optionally, a balloon or leaky balloon or fabric bag is inserted to hold at least some of the cement, and the cement is injected into the balloon. Optionally, the balloon is biodegradable, to allow bone growth through it. In an alternative embodiment, the deformer is left in place as an implant Optionally, cement is injected through the deformer. Optionally, a smaller amount of cement is required and there is optionally a reduced danger of leakage. Optionally, the implant is biodegradable, at least in part. In an alternative embodiment of the invention, the deformer is provided inside a balloon or fabric bag and used to expand the vertebra. Cement or bone slurry are optionally used to replace or as an addition to the deformer, which is optionally removed.

In an exemplary embodiment of the invention, a pliable deformer is used. Alternatively or additionally, a stiff deformer, for example made of titanium, is used.

In a different vertebral treatment method, a deformer is implanted into an inter-vertebral space, to serve as a cushion or disc replacement or disc nucleus replacement between two vertebrae.

An aspect of some embodiments of the invention relates to a system for deforming a deformer. In an exemplary embodiment of the invention, the system includes a rod on which the deformer is mounted and attached at a distal end thereof. A pusher tube pushes a second end of the deformer towards the distal end. An overtube retracts so that selected parts of the deformer are unrestrained to expand. Optionally, a first part of the deformer is exposed and the pullback of the overtube is delayed until that first part deforms. Optionally, the pushing is mechanically coupled to retraction of the overtube and includes a mechanical delaying mechanism.

In an exemplary embodiment of the invention, a last delivery step of the deformer comprises tightening the deformer so that its radial resistance increases. In an exemplary embodiment of the invention, this is achieved by axially compressing the deformer a final amount Possibly, this allows a final radial force to be applied in concert by an elongate section of the deformer, rather than by short sections one at a time.

In a non-implant configuration, the system optionally includes a mechanism for controlling the length of deformed deformer. In one example, retraction of the overtube is stopped when a desired length is achieved.

In an exemplary embodiment of the invention, the deformer is cannulated to allow cement flow therethrough. Alternatively or additionally, the system itself and/or the rod on which the deformer is mounted, is/are cannulated to allow such flow. Optionally, the rod includes a lumen, for example, for a guide wire.

In an implant configuration, the system optionally includes a mechanism for locking and releasing a deformer implant.

Optionally, the system can be used for un-deforming the deformer, for example, by pulling back on the pusher, which is optionally attached to the proximal end of the deformer. Optionally, such un-deforming does not return the overtube to its original position, thereby preventing reuse of the system.

Optionally, the system is flexible, for example for use in an endoscope. Optionally, a pull wire is used rather than a pusher, to deform the deformer. For example, the pull wire can be mounted on a pulley to pull the proximal part of the deformer towards its distal part.

Optionally, a hydraulic mechanism is used for pushing the pusher and retracting the over tube. Optionally, a hydraulic column is used to push the proximal end of the deformer and deform it.

An aspect of some embodiments of the invention relates to a shortened delivery system. In an exemplary embodiment of the invention, a handle for applying manual force to be delivered is on a side of the delivery system. In an exemplary embodiment of the invention, a pusher used for applying force to a deformer is folded or curved, so it takes up less of an axial length of the delivery system. In an exemplary embodiment of the invention, a pulling mechanism is used, for example, a mechanism including a wire and pulley. In an exemplary embodiment of the invention, the maximum dimension of the delivery system (outside of a delivery tube section thereof is less than 40 cm, less than 30 cm, less than 20 cm, less than 15 cm or a smaller, intermediate or larger value. Optionally, use is made of the fact that in some embodiments of the invention, the overtube only retracts a small amount during deformation.

An aspect of some embodiments of the invention relates to a coupling spacer. In an exemplary embodiment of the invention, a delivery system is coupled to a delivery cannula, by a spacer positioned between or extending from the delivery system body and a cannula which reaches a bone structure. The spacer optionally prevents forward and/or backward motion of the delivery system relative to the cannula, depending on the embodiment. In an exemplary embodiment of the invention, the prevention of forward motion is used to prevent deforming and/or undeforming processes inside the vertebra from pulling the delivery system forward towards the vertebra Instead, the cannula is coupled to the delivery system by the spacer and forward motion is prevent by the resting of the cannula on bone. This type of space may also be used for other intra- or inter- vertebral devices, such s balloons.

An aspect of some embodiments of the invention relates to a balloon system for pushing apart end plates of a vertebra In an exemplary embodiment of the invention, the system comprises an outer balloon and an inner balloon. The inner balloon is inflated first, pushing apart the plates and/or also limiting the expansion of the outer balloon to at least include the shape of the inner balloon. It is expected that the inner balloon should be able to, in most cases, expand properly, thereby preventing migration of the outer balloon and/or setting a limit on the sideways extent of the outer balloon. Optionally, the balloons are inflated in a staggered manner. Optionally, a third or additional balloons are provided enclosing the first and second balloons.

An aspect of some embodiments of the invention relates to applying a desired displacement inside the body. Optionally, the displacement is applied with some degree of compliance to body geometry, while not allowing the displacement to be diverted to a different direction as a balloon might. Optionally, the displacement is applied with an increase in diameter of a displacing device, for example, of at least 50%, 100%, 150%, 200%, 300%, or any intermediate or greater value. In an exemplary embodiment of the invention, the desired displacement is achieved to within 5 mm, 3 mm, 2 mm, 1 mm or better using a pre-determined amount of distortion of the deformer.

In an exemplary embodiment of the invention, the displacement is applied in one direction while a sensitive tissue lies in a second direction. Optionally, the displacement is directed to not damage the sensitive tissue.

There is thus provided in accordance with an exemplary embodiment of the invention, a medical grade deformer, comprising:

an axial member; and

a pliable tube mounted on said axial member and adapted to be deformed from a first, narrower diameter, configuration to a second, greater diameter, configuration. Optionally, said tube is slotted through its thickness. Alternatively, said tube is not slotted.

In an exemplary embodiment of the invention, said deformer comprises at least one end engaging one end of said tube and adapted to apply compressive force to said tube for achieving said deformation. Optionally, said deformer comprises at least a second end one end engaging a second end of said tube and adapted to cooperate with said first end to compress said tube. Optionally, said deformer comprises said two engaging ends and said axial member lock to maintain said pliable tube in a greater diameter configuration.

In an exemplary embodiment of the invention, said deformer comprises said tube changes configuration by axial compression thereof.

In an exemplary embodiment of the invention, said axial member is rigid.

In an exemplary embodiment of the invention, said axial member is flexible.

In an exemplary embodiment of the invention, said axial member extends out of said tube and is attached to a handle.

In an exemplary embodiment of the invention, said axial member comprises a release mechanism for release of said deformer from a delivery system. Optionally, said axial member comprises a locking mechanism for locking of said deformer in a greater diameter configuration in conjunction with release.

In an exemplary embodiment of the invention, said deformer includes a channel adapted for bone filler flow.

In an exemplary embodiment of the invention, said channel is formed in said axial member. Alternatively or additionally, said channel is formed between said axial member and said tube.

In an exemplary embodiment of the invention, said axial member extends from said tube and is adapted to function as a hinge of a joint.

In an exemplary embodiment of the invention, said deformer forms a bone attachment unit for a prosthesis.

In an exemplary embodiment of the invention, said deformer comprises an enclosing bag, which surrounds said tube in said second configuration. Optionally, said bag is bio-degradable in the body. Alternatively or additionally, said bag is porous.

In an exemplary embodiment of the invention, said deformer defines a general volume in the shape of a cylinder when in said second configuration.

In an exemplary embodiment of the invention, said deformer defines a general volume in the shape of a truncated pyramid when in said second configuration.

In an exemplary embodiment of the invention, said deformer defines an axially rotationally asymmetric general volume when in said second configuration.

In an exemplary embodiment of the invention, said deformer defines a predetermined general volume when in said second configuration.

In an exemplary embodiment of the invention, said deformer comprises a set of axially contiguous zones with different material properties.

In an exemplary embodiment of the invention, said deformer has a non-smooth outer surface in said second configuration.

In an exemplary embodiment of the invention, said deformer is stiff enough, when in said second configuration to resist a transaxial force of at least 50 Kg.

In an exemplary embodiment of the invention, said deformer, when in said second configuration has an axial applied force of at least 2 Kg.

In an exemplary embodiment of the invention, said pliable material has a shore hardness of between 50 A and 90 D.

In an exemplary embodiment of the invention, said pliable material is nonmetallic.

In an exemplary embodiment of the invention, said pliable material is polymeric.

In an exemplary embodiment of the invention, said deformer includes at least one axial thread.

In an exemplary embodiment of the invention, said deformer includes at least one circumferential thread.

In an exemplary embodiment of the invention, said deformer, in said second configuration, defines a general volume and wherein said deformer fills at least 30% of said volume.

In an exemplary embodiment of the invention, said deformer, in said second configuration, defines a general volume and wherein said deformer fills at least 50% of said volume.

In an exemplary embodiment of the invention, said tube defines a plurality of slots, such that when deformed to the second configuration, a plurality of axially displaced leaves extend from said tube to define said second configuration. Optionally, said tube defines at least three axially displaced leaves. Alternatively or additionally, adjacent leaves support each other, in said second configurations.

In an exemplary embodiment of the invention, an end leaf is shorter than a non-end leaf.

In an exemplary embodiment of the invention, an end leaf is supported, on one side thereof, by an end cap of said deformer.

In an exemplary embodiment of the invention, adjacent leaves deform each other.

In an exemplary embodiment of the invention, at least 50% of the leaves are deformed from a plane.

There is also provided in accordance with an exemplary embodiment of the invention, a deformer, comprising a non-inflatable substantially non-absorbent deformable non-metallic body having two configurations, a first configuration in which said body has a narrower diameter and a second configuration in which said narrower diameter is greater, wherein said deformer is adapted to remain substantially undeformed under a force of over 10 Kg and wherein said deformer is sized for positioning inside a human vertebra. Optionally, said deformer is adapted to remain substantially undeformed when in a human lumbar vertebra in standing condition. Alternatively or additionally, said deformer is self-expanding. Alternatively or additionally, the deformer is provided as part of kit including a spinal access tool.

There is also provided in accordance with an exemplary embodiment of the invention, a method of spinal surgery, comprising:

inserting a non-inflatable non-absorbent deformable deformer into a vertebra; and

deforming said deformer such that cortical bone of vertebral faces of said vertebra, move relative to each other.

There is also provided in accordance with an exemplary embodiment of the invention, a method of treating a bone, comprising:

inserting a unsealed pliable element into the bone; and

mechanically deforming the pliable element such that said pliable element applies deforming force on the bone. Optionally, said pliable element comprises at least one open aperture of cross-section greater than 0.5×0.5 mm. Alternatively or additionally, said bone comprises a vertebral bone. Alternatively, said bone comprises a long bone.

There is also provided in accordance with an exemplary embodiment of the invention, a method of achieving a desired bone displacement, comprising:

determining a desired degree of displacement;

determining a deformation amount, of a deformer, suitable to achieve said deformation;

inserting a suitable deformer into a bone; and

deforming said deformer, said deformation amount, to achieve said displacement to within 2 mm.

There is also provided in accordance with an exemplary embodiment of the invention, a method of deforming a medical deformer, comprising:

(a) applying a compressing force;

(b) retracting an overtube;

(c) repeating (a) and (b) such that a plurality of sections of the deformer deform to a greater diameter; and

(d) applying a final compressing force to stiffen the deformer. Optionally, said repeating is intermittent.

There is also provided in accordance with an exemplary embodiment of the invention, an inflatable bone moving element, comprising:

(a) a first balloon;

(b) a second enclosing balloon; and

(c) a dual balloon inflator adapted to first inflate the inner balloon and then inflate the outer balloon, such that the inner balloon constrain the direction of expansion of the outer balloon.

There is also provided in accordance with an exemplary embodiment of the invention, a deformer delivery system, comprising:

a distal end adapted to be inserted into a vertebra through a cannula;

a proximal body including a handle; and

a spacer adapted to vary in length and maintain a distance between said body and said cannula, thereby maintaining a relative position of said distal end and said vertebra. Optionally, said spacer is integral to said system.

There is also provided in accordance with an exemplary embodiment of the invention, a deformer delivery system, comprising:

(a) an over tube;

(b) an over tube retractor;

(c) a pushing element adapted to deform a deformer; and

(d) a synchronizing mechanism adapted to retract said overtube in synchrony with advancing said pushing element, wherein said retractor delays until after said pushing element starts deforming said deformer a given amount.

There is also provided in accordance with an exemplary embodiment of the invention, a deformer delivery system, comprising:

(a) an over tube;

(b) an over tube retractor;

(c) a pushing element adapted to deform a deformer; and

(d) a synchronizing mechanism adapted to retract said overtube in synchrony with advancing said pushing element, wherein said retractor is adapted to retracts said overtube also when said pushing element is retracted.

There is also provided in accordance with an exemplary embodiment of the invention, a deformer delivery system, comprising:

(a) an over tube;

(b) an over tube retractor;

(c) a pushing element adapted to deform a deformer; and

(d) a synchronizing mechanism adapted to retract said overtube in synchrony with advancing said pushing element, wherein said delivery system has an axial extent shorter than 130% of an extent of motion of said pushing element. Optionally, said axial extent is less than 100% of said extent of motion.

### BRIEF DESCRIPTION OF THE FIGURES

Particular embodiments of the invention will be described with reference to the following description of exemplary embodiments in conjunction with the figures, wherein identical structures, elements or parts which appear in more than one figure are optionally labeled with a same or similar number in all the figures in which they appear, in which:

FIG. 1A is a schematic illustration of an undeformed deformer, in accordance with an exemplary embodiment of the invention;

FIGS. 1B-1H are schematic illustrations of a deformer during deformation, in accordance with an exemplary embodiment of the invention;

FIGS. 2A-2E are schematic axial cross-sectional views showing the deformation of the deformer of FIG. 1A, in accordance with an exemplary embodiment of the invention;

FIGS. 3A-3E show steps in the treatment of a vertebra, in accordance with an exemplary embodiment of the invention;

FIG. 4A is a flowchart of a method of treating a vertebra in accordance with FIGS. 3A-3E;

FIGS. 4B-4H are parts of a kit for treating a vertebra, in accordance with an exemplary embodiment of the invention;

FIGS. 5A-SD are schematic views of a delivery system, in accordance with an exemplary embodiment of the invention;

FIG. 6 is a schematic cross-sectional view of a hydraulic delivery system in accordance with an exemplary embodiment of the invention

FIG. 7A is a schematic cross-sectional view of a belt based delivery system in accordance with an exemplary embodiment of the invention

FIGS. 7B and 7C illustrate a wire based delivery system, in accordance with an exemplary embodiment of the invention;

FIGS. 7D and 7E illustrate a soft-material based delivery system, in accordance with an exemplary embodiment of the invention;

FIG. 7F illustrates a flexible deforming system, in accordance with an exemplary embodiment of the invention;

FIG. 7G illustrates a ratchet mechanism for the system of FIG. 7F, in accordance with an exemplary embodiment of the invention;

FIG. 7H illustrates a spacer, in accordance with an exemplary embodiment of the invention;

FIGS. 8A-8C illustrates an implant release and locking mechanism, in accordance with an exemplary embodiment of the invention;

FIGS. 9A-9F illustrate various deformer geometries in accordance with exemplary embodiments of the invention;

FIG. 10 schematically shows a spinal joint in accordance with an exemplary embodiment of the invention;

FIGS. 11 and 12 shows the use of an implanted deformer for supporting a humerus head (FIG. 11) and a tibial plateau (FIG. 12), in accordance with an exemplary embodiment of the invention.

FIG. 13 is a schematic illustration of an intra-medullar nail, in accordance with an exemplary embodiment of the invention;

FIG. 14 illustrates a hip trochanter support implant using a deforming element in accordance with an exemplary embodiment of the invention;

FIGS. 15A and 15B illustrates a dental implant in accordance with an exemplary embodiment of the invention; and

FIGS. 16A-16E show a balloon-in-balloon configuration, in accordance with an exemplary embodiment of the invention.

### DETAILED DESCRIPTION OF EXEMPLARY EMBODIMENTS

Slotted Tube Expander Design

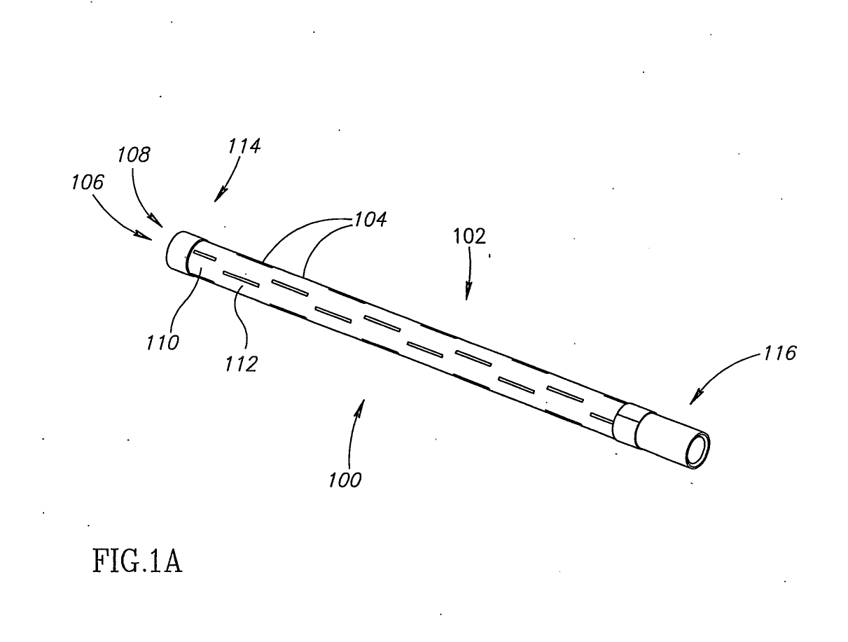

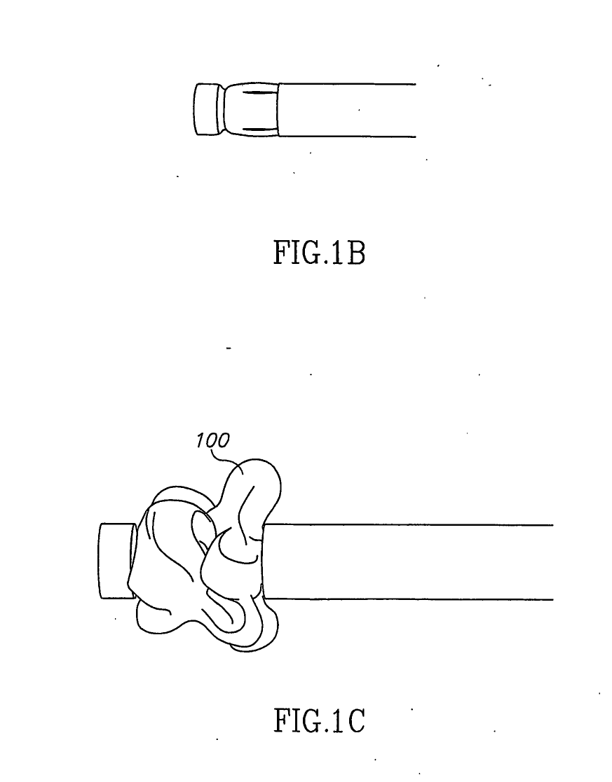

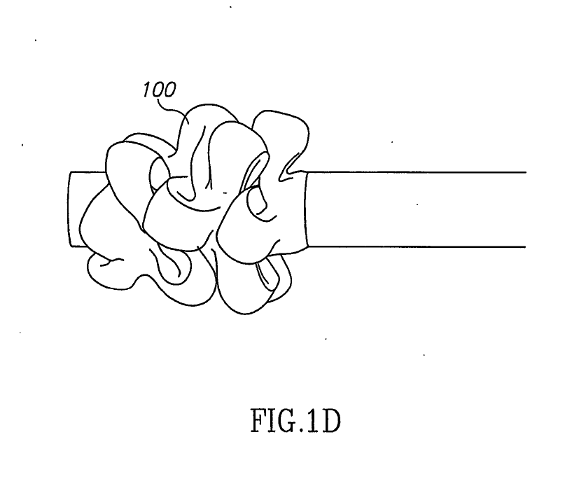

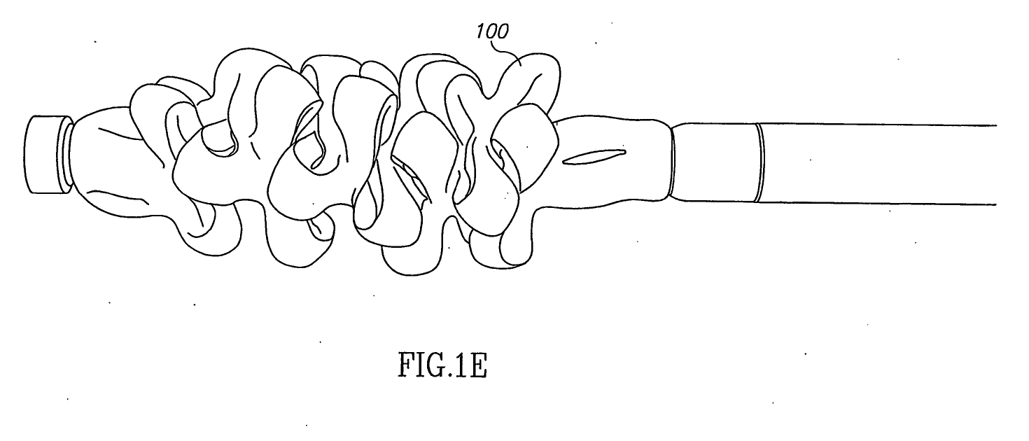

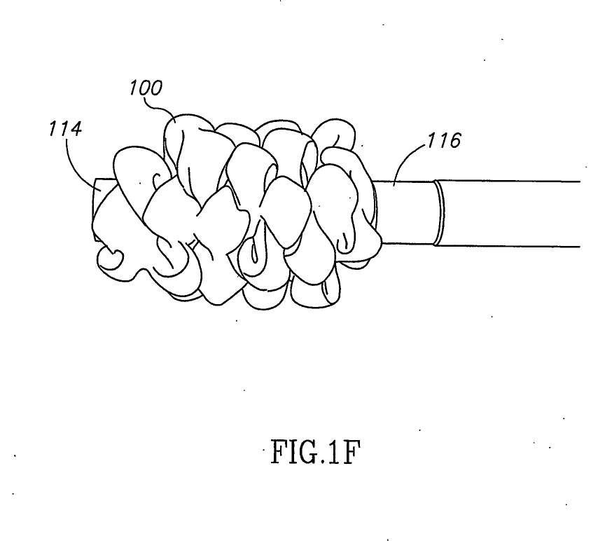

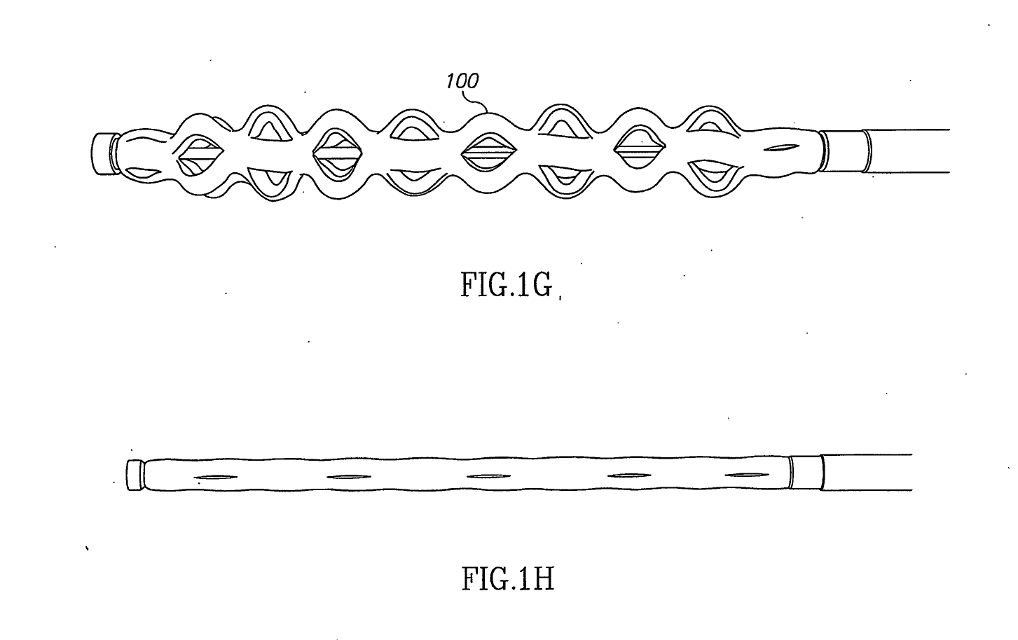

FIG. 1A is a schematic illustration of an undeformed deformer 100, in accordance with an exemplary embodiment of the invention. FIG. 1F is a schematic illustration of a fully deformed deformer 100, in accordance with an exemplary embodiment of the invention. FIGS. 1B-1E show intermediate states and FIGS. 1G-1H show an undeforming process (described below).

In this design, deformer 100 comprises a tube body 102, having a plurality of slots 104 formed along its length, in an axial direction. In the embodiment shown, slots 104 are arranged in alternating lines 106 and 108 such that a plurality of alternating leaf lines are formed, comprising leaves 110 alternating (axially and radially) with leaves 112. As will be described below, for example, other designs may be provided. A distal end 114 and a proximal end 116 are also marked on FIG. 1A.

As can be seen in FIG. 1F, when fully deformed, deformer 100 substantially fills a volume of space (e.g., a more or less predefined volume formed when the deformer is deformed). Also, as shown, the leaves support each other. Optionally, two neighboring leaves touch each other on at least 20%, 30%, 40%, 50%, 60% or more of their matching sides.

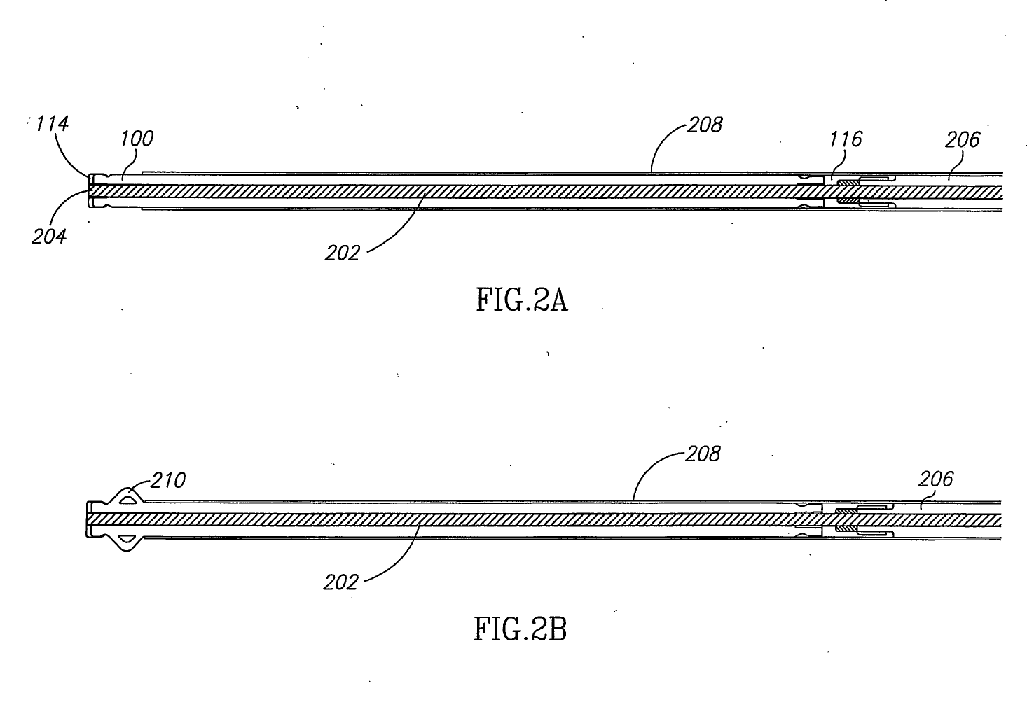

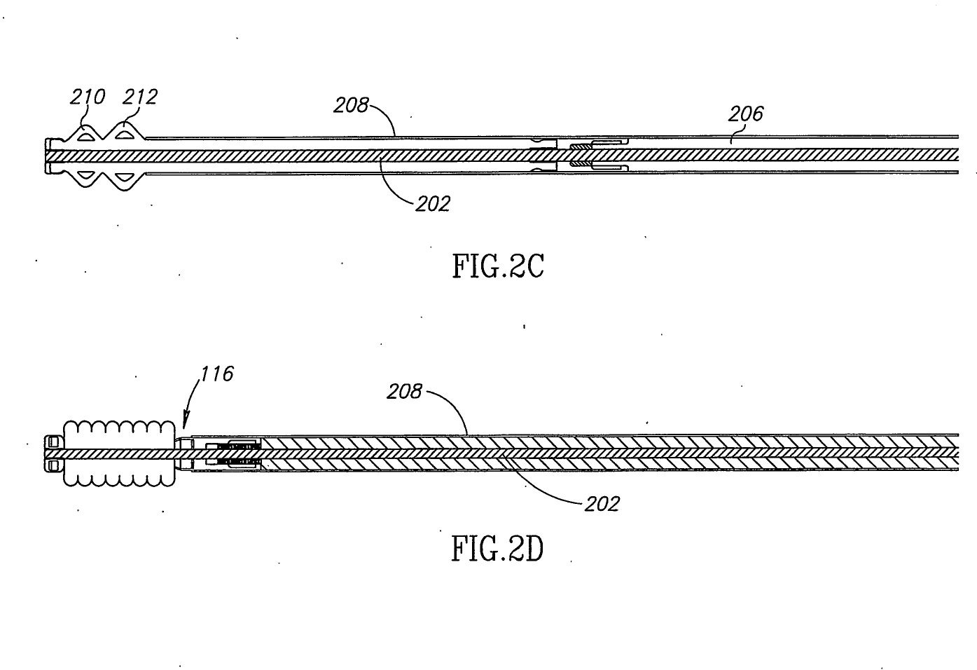

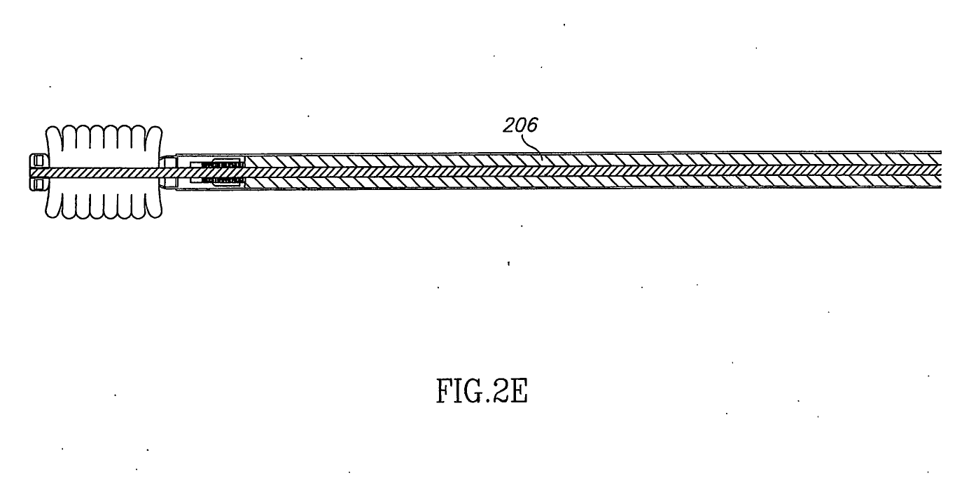

FIGS. 2A-2E are schematic axial cross-sectional views showing the deformation of the deformer of FIG. 1A, in accordance with an exemplary embodiment of the invention.

FIG. 2A shows deformer 100 mounted on a delivery rod 202 and optionally attached (e.g., at distal end 114 thereof) to an end 204 of delivery rod 202. A pusher 206, for example a tube, pushes proximal end 116 of deformer 100 towards distal end 114. An overtube 208 restrains the radial deformation of deformer 100, as will be described below.

In FIG. 2B, pusher tube 206 is advanced, while overtube 208 is not retracted. As a result, a first plurality of leaves 210 extend radially past overtube 208.

In FIG. 2C, pusher tube 206 is advanced more (for example continuously or in discrete steps) while overtube 208 is retracted (for example continuously or in (optionally matching) discrete steps). The ratio between the movements can be axially linear or non-linear, for example. As a result, a second plurality of leaves 212 extend radially past overtube 208 and optionally lean on leaves 210. As can be seen, leaves 210 and 212 are not axially compressed to the fullest extent possible. In some embodiments, as more leaves are extended out, the tips of previously extended leaves move axially. Optionally, this axial motion is used for engaging tissue or engaging a nearby deformer, for example for dual deformer use.

FIG. 2D shows the state after all the leaves have been deformed out. It should be noted that the process of leaf deformation may appear in some embodiments like an extrusion, even though the deformer is not flowing.

FIG. 2E shows the effect of further axial pushing by pusher 206 once all the leaves have been extended. The result shown is an axial compression and radial stiffening of the leaves. Optionally, this simultaneous radial stiffening of multiple locations along the deformer, allows force to be applied to nearby tissue in a desired direction.

Referring back to FIG. 1F, it should be noted that in some cases not all of the leaves are extended in a perfect manner. However, in many applications this is not a problem. In some applications, the pliability of the material allows the other leaves to adjust their position accordingly. In some applications, the imperfect extension allows space for cement and/or other fluids.

Reference is now made in more detail to FIGS. 1B-1H. FIG. 1B shows deformer 100 before any leaves are extended. FIG. 1C shows deformer 100 after four sections of leaves are extended. FIG. 1D shows deformer 100 after some more leaves are extended. FIG. 1E shows deformer 100 with nearly all the leaves extended. FIG. 1F shows deformer 100 with all the leaves extended and with axial compression.

FIG. 1G shows deformer 100 during removal, due to retraction of pusher 206. FIG. 1H shows deformer 100 fully un-deformed so it can be removed from the body.

In some embodiments of the invention, deformer 100 is deformed all at once, without an overtube or without gradual retraction thereof. Suitable treatment or design of the deformer may, however, impose an order on the deformation, for example, weaker sections may tend to deform first.

Vertebral Treatment System

In an exemplary embodiment of the invention, a deformer is used to treat compression fractures of the vertebrae, by inserting a deformer in a narrow diameter configuration, into a vertebra and deforming the deformer such that it expands the vertebra.

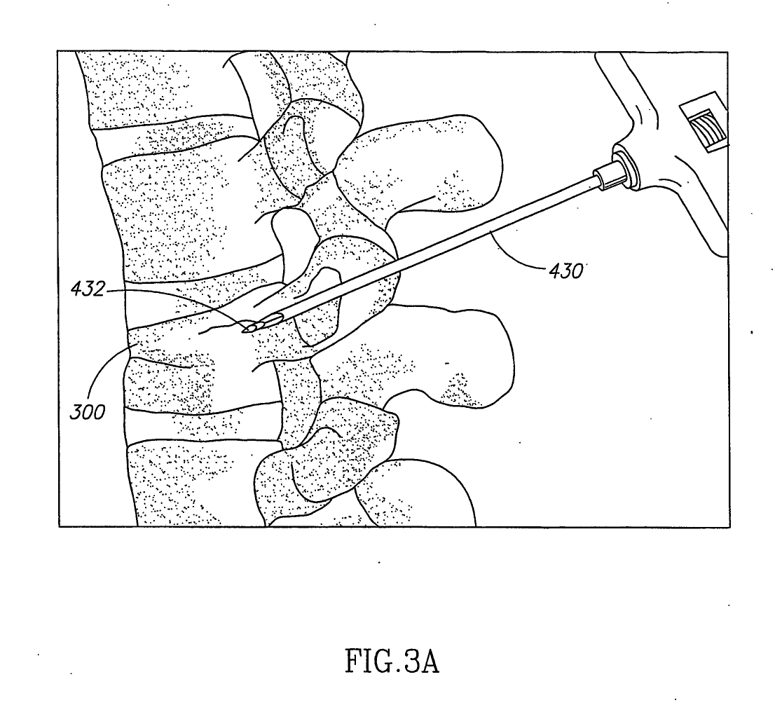

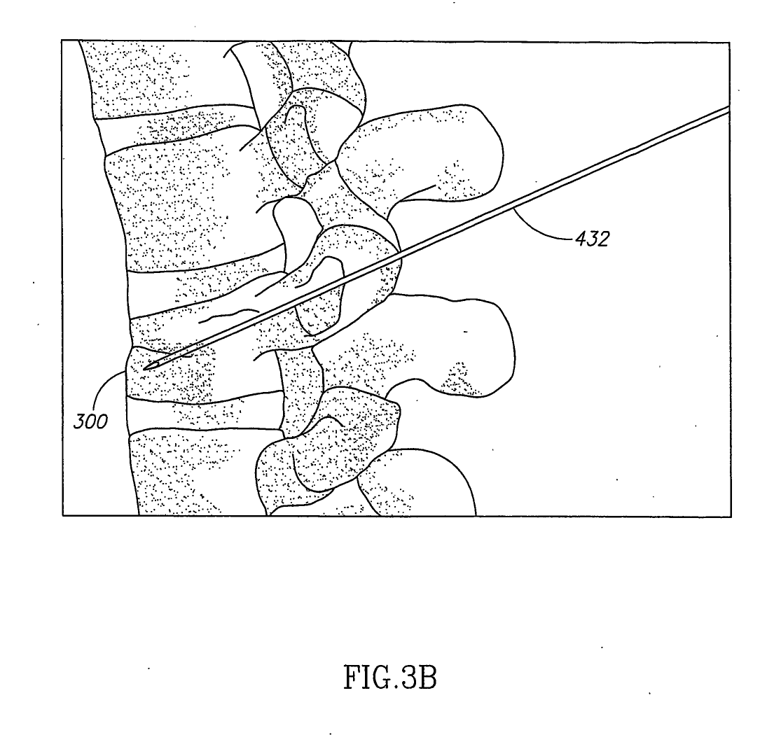

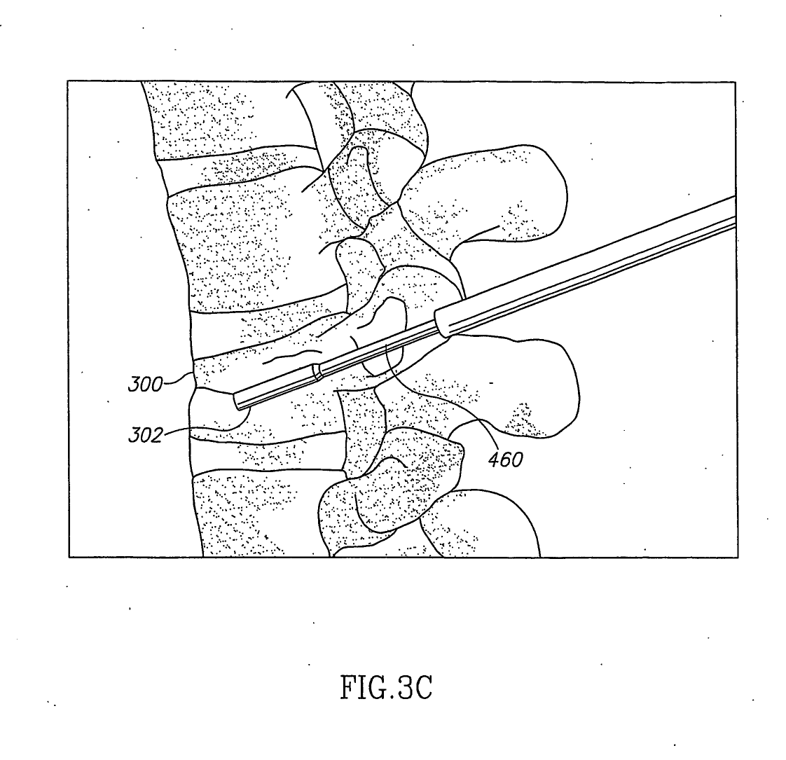

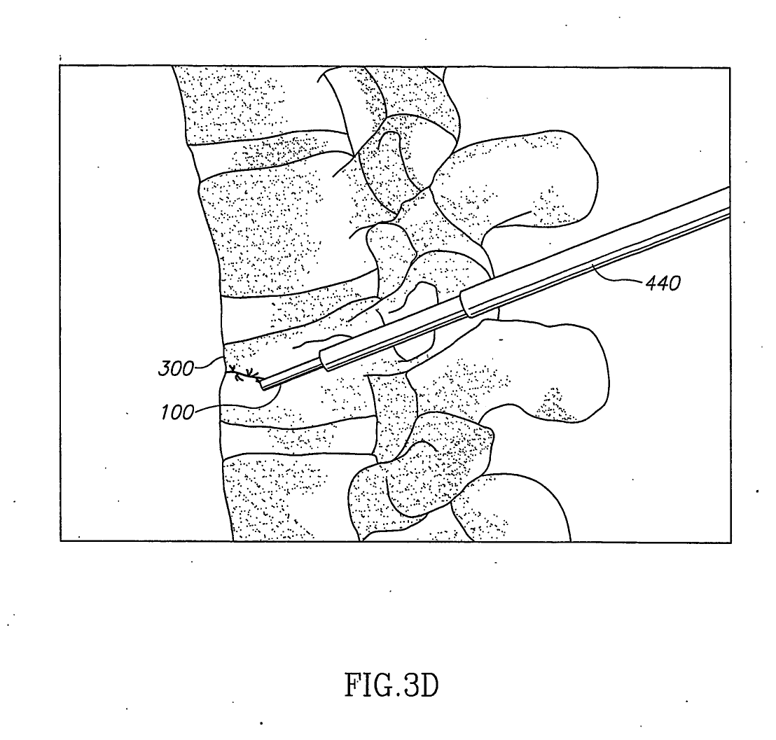

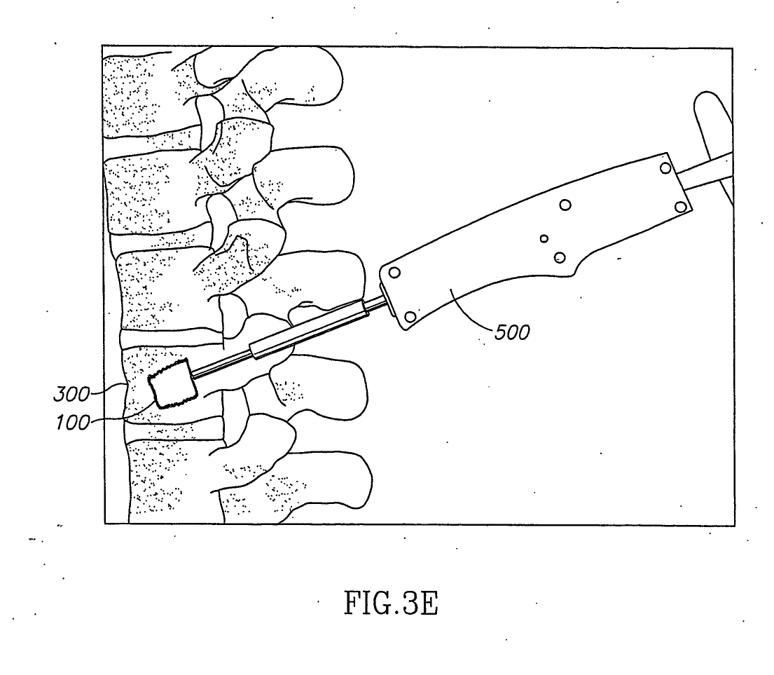

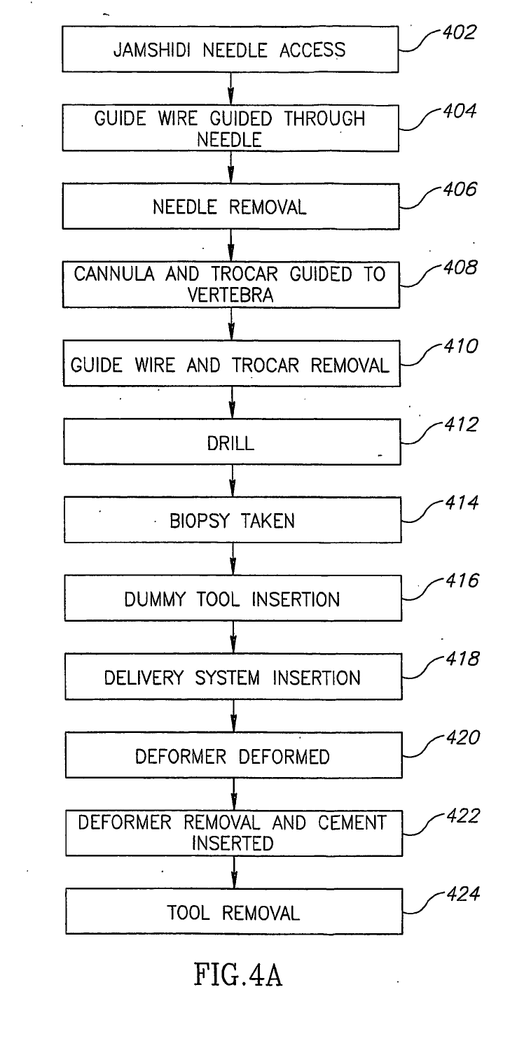

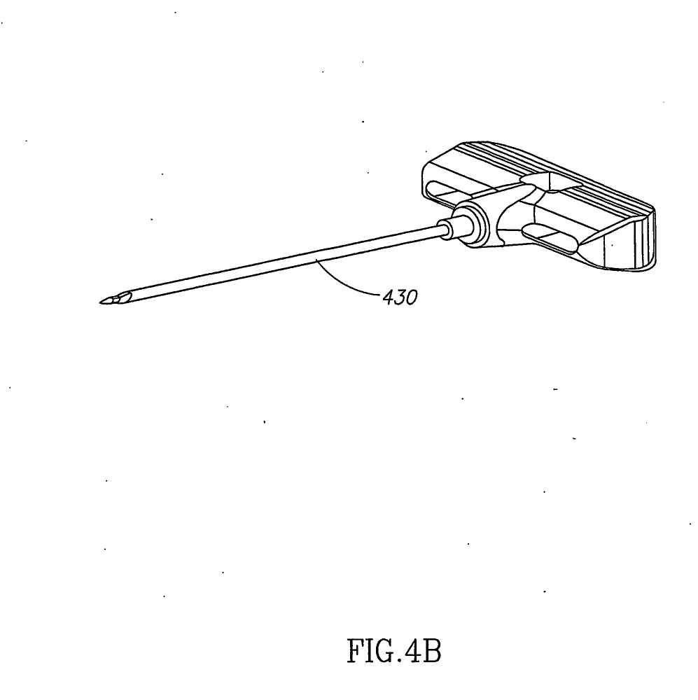

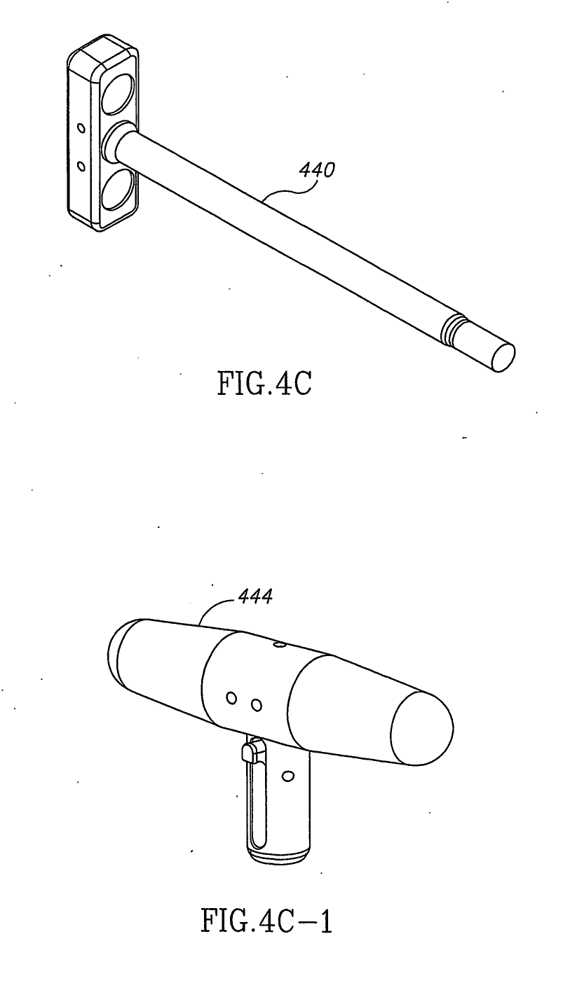

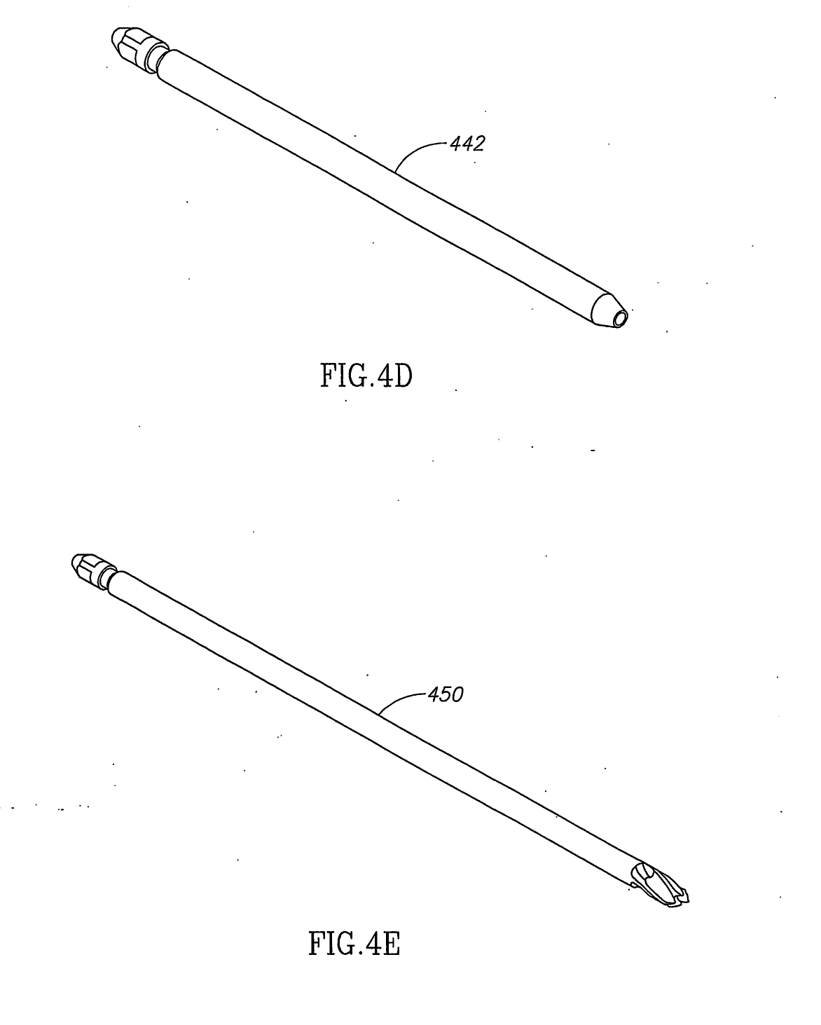

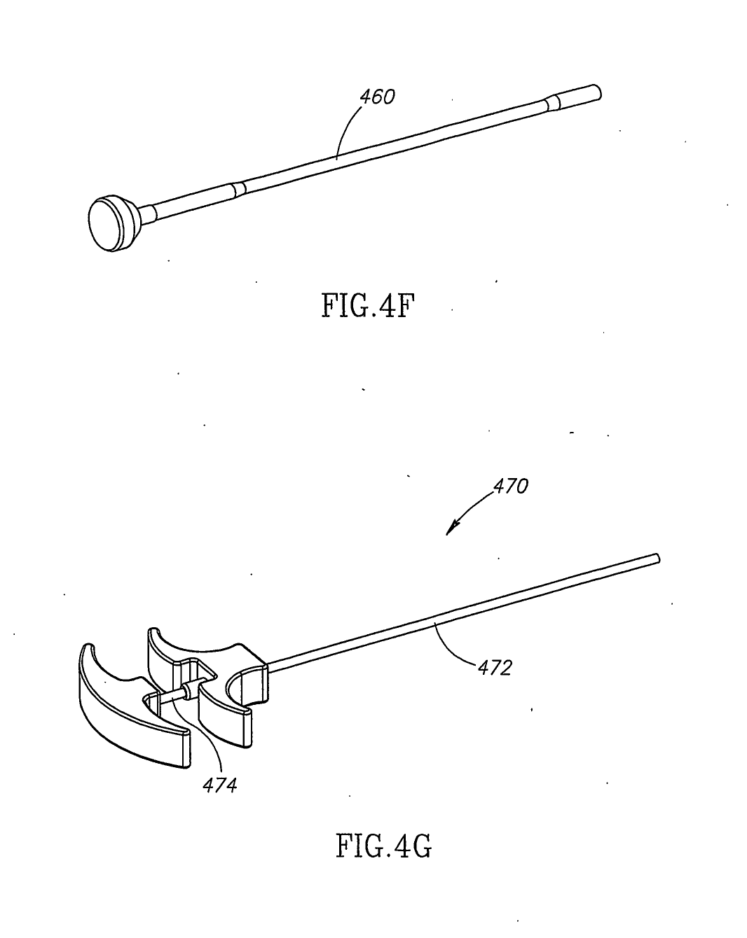

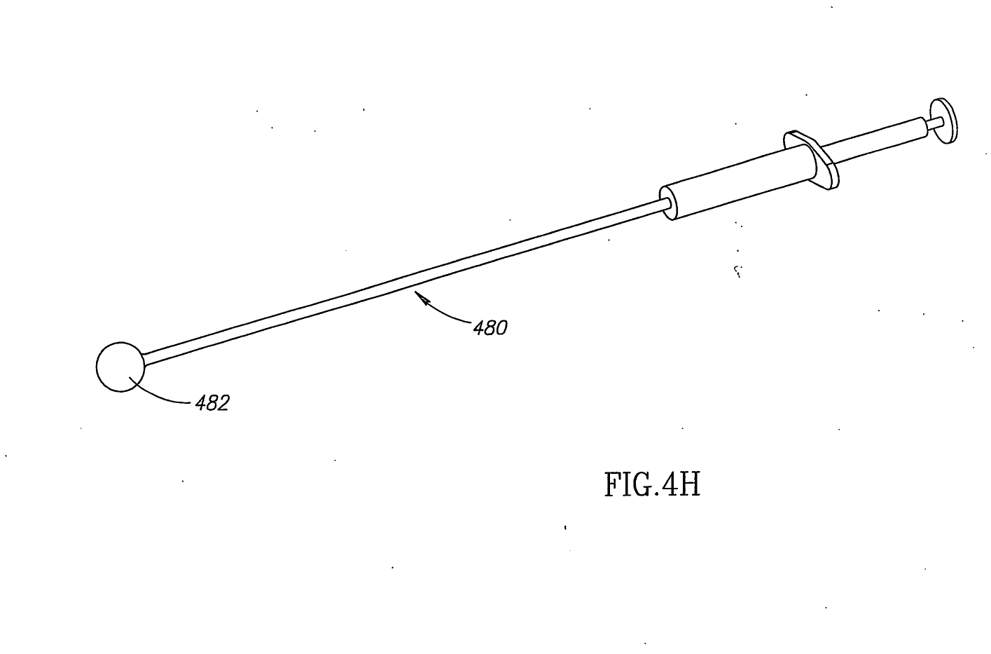

FIGS. 3A-3E show steps in the treatment of a vertebra, in accordance with an exemplary embodiment of the invention. FIG. 4A is a flowchart 400 of a method of treating a vertebra in accordance with FIGS. 3A-3E. FIGS. 4B-4H are parts of a kit for treating a vertebra, in accordance with an exemplary embodiment of the invention. Other kits may include fewer or greater number of elements.

Referring to FIG. 4A. At 402, a vertebra 300 (FIG. 3A) is accessed using, for example, a Jamshidi needle 430 (FIG. 4B), optionally with an inner stylet or guidewire (which are optionally removed later).

Alternatively, if no guidewire was used earlier, at 404, a guide wire 432 (FIG. 3B) is guided through needle 430 into vertebra 300.

At 406, needle 430 is removed (FIG. 3B). As can be appreciated, other methods of accessing a vertebra can be used and many are known in the art. In addition, it is not essential to use a guidewire for accessing the vertebra and other means can be used to guide deformer 100 to its target.

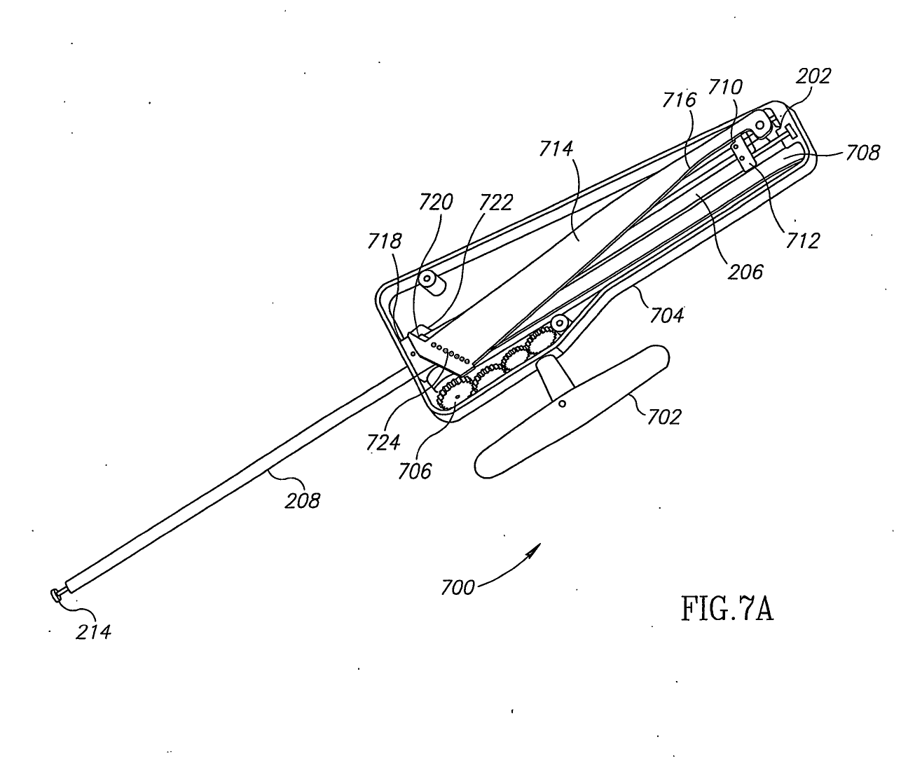

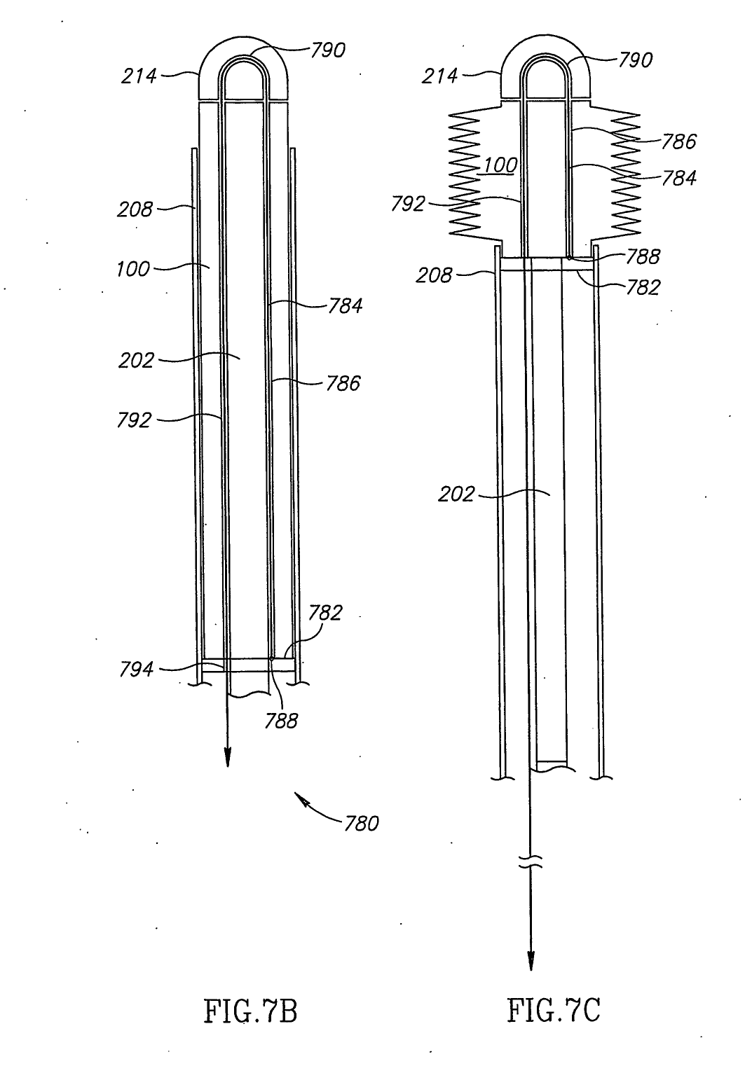

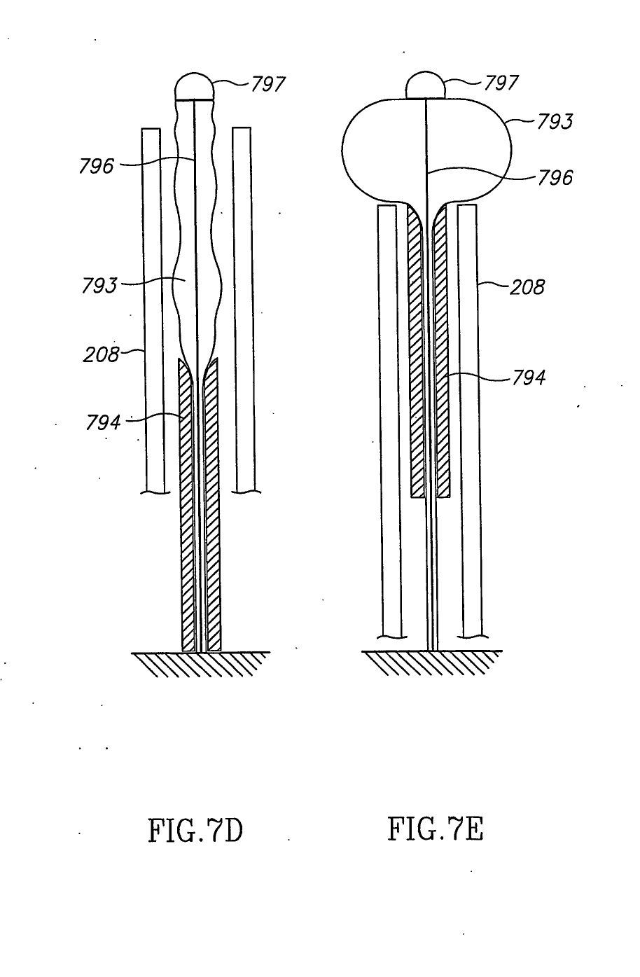

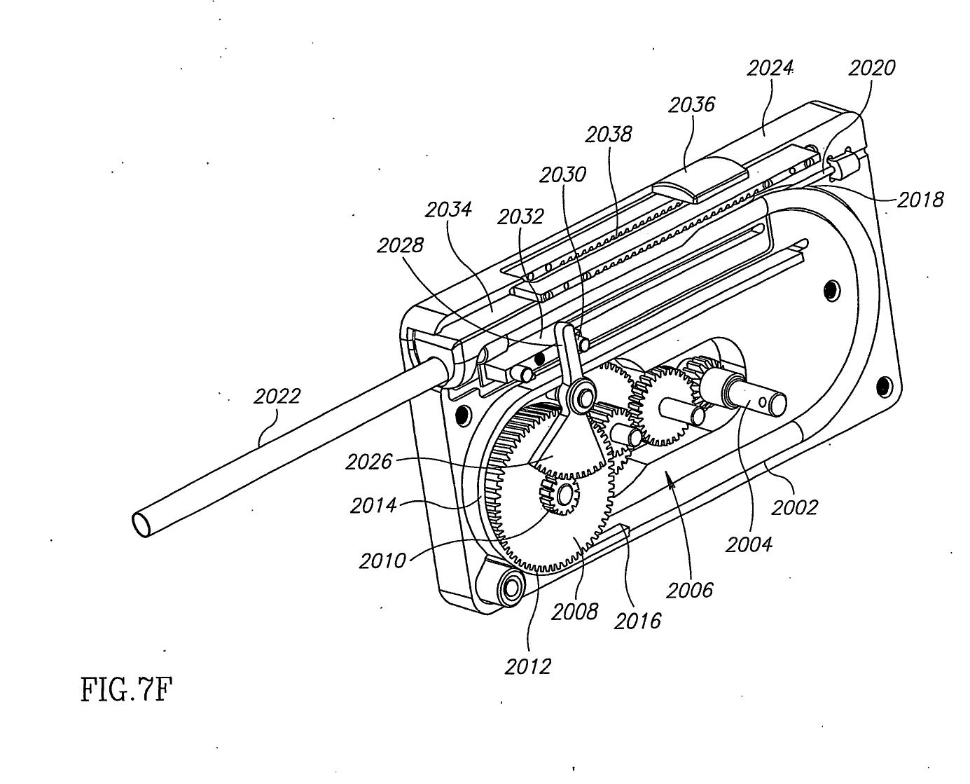

At 408, a cannula 440 (FIG. 4C), with an optional trocar 442 (FIG. 4D) are guided along guidewire 432 to vertebra 300. A handle 444 (FIG. 4C-1) optionally couples the cannula and trocar 442 and/or can be attached to a drill or other tools (e.g., a modular handle). Once inserted into vertebra 300, cannula 440 is optionally rotated to ensure it engages vertebra 300 and/or nearby bones. Alternatively or additionally, other fixation methods, for example, forward pressure or sideways extending leaves or a balloon, are used to hold cannula 440 in place. A spacer (FIG. 7H) is optionally positioned between cannula 440 and the housing of the delivery system (e.g., 2024, FIG. 7F), to restrict anterior displacement of the delivery system during deformer deforming and/or undeforming.

At 410, guide wire 432 and/or trocar 442 are removed, leaving only cannula 440 within the body. Optionally, trocar 442 is removed and then a cannulated drill is inserted. Optionally, the guide wire is removed following partial drilling, and end of the drilling is performed without the guide wire, to prevent its anterior advancement

At 412, a drill 450 (FIG. 4E) is used to ream out a section of vertebra 300 and form a void 302 (shown in FIG. 3C) for deformer 100. Optionally, the drill is guided along guidewire 432. As can be seen, drilling may be practiced at various steps of the process, or not at all, depending on the exact implementation.

At 414, a biopsy is optionally taken. Alternatively, a biopsy is taken earlier, for example at 402 or 404.

At 416, a dummy tool 460 (FIG. 4F) is optionally inserted and x-rays are acquired to verify its placement (FIG. 3C). Other verification means may be used as well, such as ultrasound and/or position sensing. Optionally, the verification is used to select a deformer diameter and/or length.

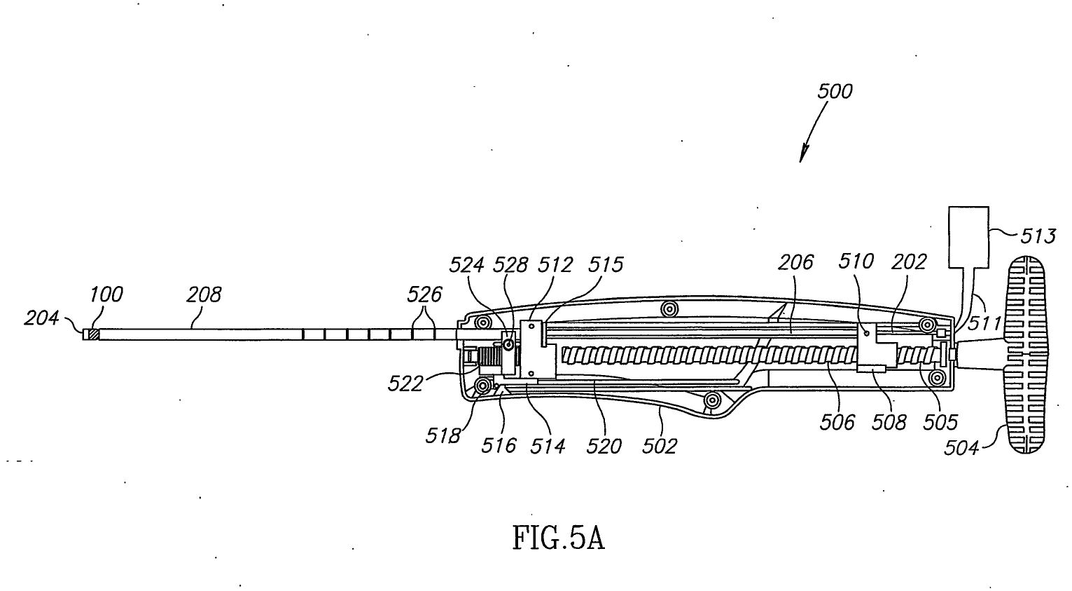

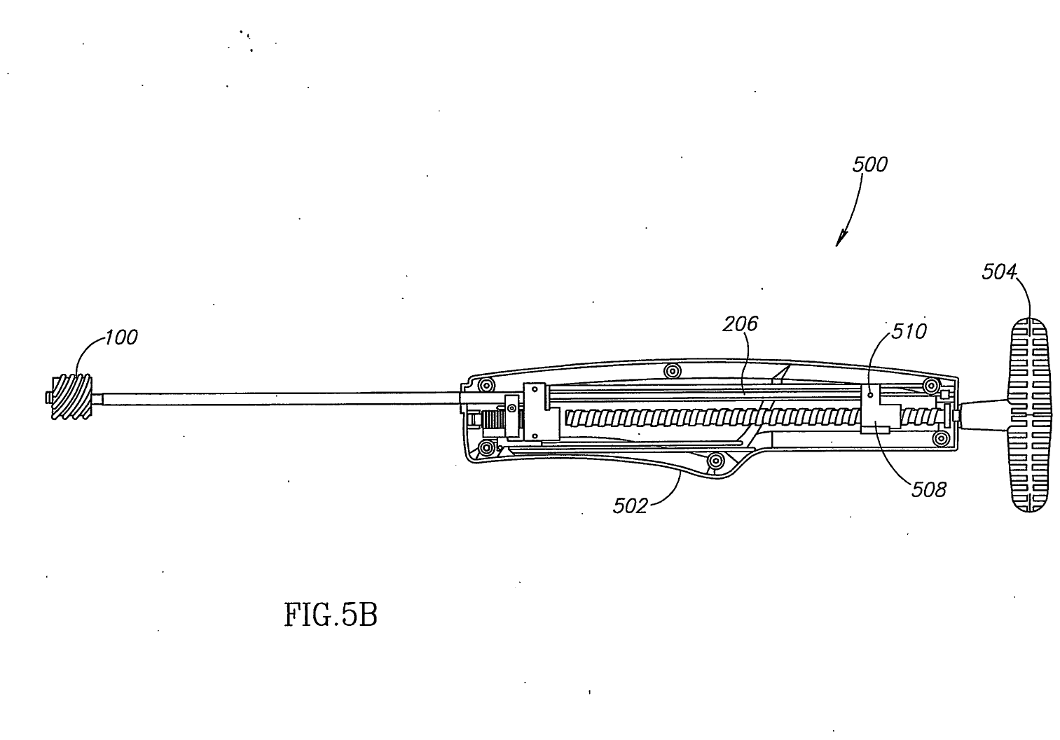

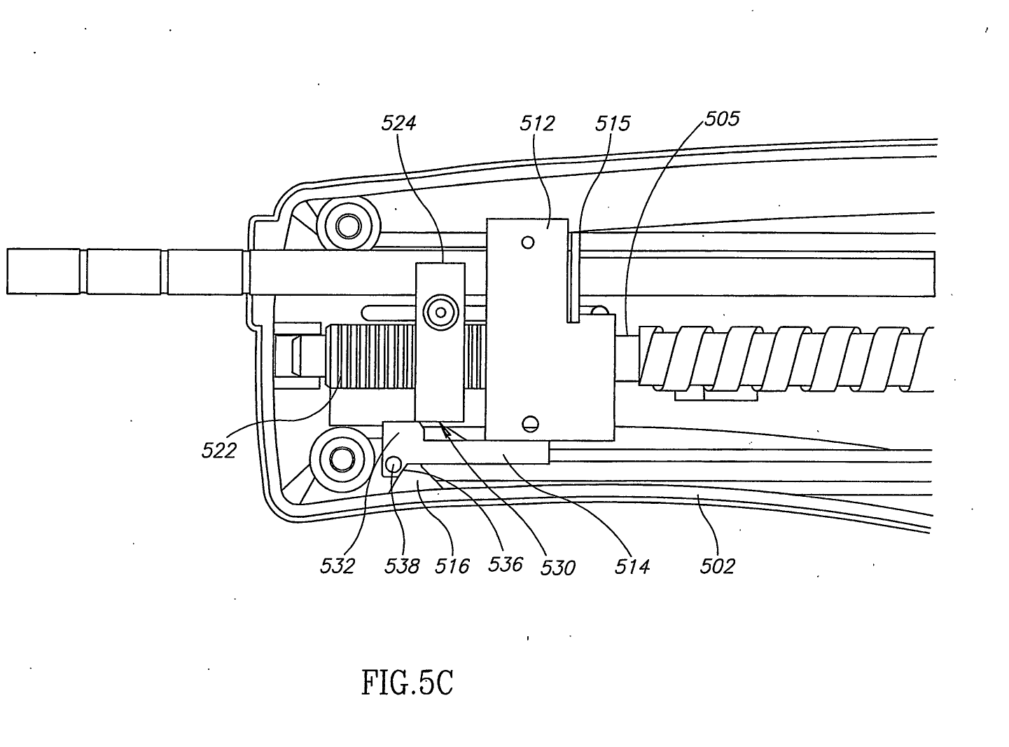

Dummy tool 460 is then removed, and at 418, deformer 100, mounted on a delivery system 500 (FIG. 5) is inserted into void 302 (FIG. 3D). It should be appreciated that a deformer can be inserted into a vertebra from various directions. Further, multiple deformers can be inserted into a same vertebra from multiple direction, for example posterior, or lateral or postero-lateral approaches and/or from either side of the body plane, in either approach.

At 420, deformer 100 is deformed (FIG. 3E). During the deformation, a determination of the actual deformation of vertebra 300 may be acquired, to ensure correct expansion and/or prevent over expansion and/or fracturing damage to vertebra 300. For example, x-ray or CT images may be acquired.

At 422, deformer 100 is optionally removed and a cement delivery tube 470 (FIG. 4G) is inserted instead. Delivery tube 470, in one embodiment, includes an outer tube section 472 and an inner plunger 474. Cement, stored in a lumen of outer tube 472 is forced by the plunger into void 302. Exemplary materials which may be injected include, bone chips, bone slurry (e.g., auto-graft, xenograf, allograft, from cadavers), PMMA, calcium phosphate and/or calcium sulfate.

At 424, all the tools are removed and the procedure is completed. Surgical holes, etc. may be closed using methods known in the art.

In an alternative procedure, deformer 100 is released at the end of act 420 and remains in the body. Optionally, deformer 100 is mounted on a cannulated rod (e.g., rod 202 with an inner bore, a proximal inlet and one or more distal exit holes) through which cement or other materials can be provided to void 302. Alternatively or additionally, the cement is provided via an over tube (not shown) which surrounds rod 202 (FIG. 2A) and has a diameter small enough to reach into the vertebra, so cement will not leak out.

In an alternative procedure, cement delivery tube 470 is replaced by a balloon delivery element 480 (FIG. 4H), which expands a balloon 482 inside void 302, to maintain the shape of vertebra 300. The expansion can be, for example, with cement, with a fluid, such as saline and/or with particle matter, such as bone fragments. Balloon 482 is optionally biodegradable in the body, for example being made of Poly(L-Lactide-co-capralactone) 70:30 or Poly(L-Lactide-co-glycolide) 85:15, 82:18 or 10:9. Optionally, a mesh is used instead of a balloon, to allow leakage of some bone cement.

In an alternative procedure, a balloon and deformer delivery system is used, in which a deformer deforms inside a balloon. Optionally, balloon is biodegradable. Optionally, cement and/or other materials are provided through a channel provided in the delivery system.

Optionally, only a biodegradable balloon is used without an internal deformer. Sufficient fluid pressure is optionally provided, for moving the cortical plates.

Details of Exemplary Deformer Delivery System

FIGS. 5A-5D illustrate a deformer delivery system 500 in accordance with an exemplary embodiment of the invention. FIG. 5A shows delivery system 500 before deformation of deformer 100. FIG. 5B shows system 500 after deformation of deformer 100.

Delivery system 500 comprises a body 502 having a handle 504 for deforming deformer 100. In other embodiments, a power source, for example, an electric motor or a hydraulic power source are used for actuating the deformation instead of handle 504. When handle 504 is rotated, a rod 505 attached thereto is rotated. A threading 506 on rod 505 engages a matching threading or projection on a nut 508. Nut 508 is coupled to pusher 206, for example, via a pin 510. Thus, rotation of handle 504 advances pusher 206. It should be noted that in an exemplary embodiment of the invention, rod 202, running through pusher 206, does not move and maintains its end 204 in a fixed relationship to body 502, while pusher 206 is moved.

The rotation of rod 505 optionally retracts overtube 208, as well. Optionally, the retraction is delayed relative to the advancing of pusher 206. In an exemplary embodiment of the invention, the following mechanism is used. A nut 524 is mounted on a distal part of rod 505, with a threading 522 which optionally has a smaller pitch than threading 506, so retraction of overtube 208 is less pronounced than advancing of pusher 206. Overtube 208 is coupled to a block 512 that is moved by nut 524. However, in an exemplary embodiment of the invention, once retracted, overtube 208 cannot return to its starting position. For example, a ratchet mechanism may prevent such return. Optionally, a locking disk 515 allows only one way motion of block 512, relative to pusher 206.

Meeting of nut 508 and block 512 optionally stops the deforming of deformer 100. Alternatively, one or more stops (not shown) are provided to prevent motion of one or both of nuts 508 and 524 and/or block 512. Optionally, such stops are movable, by a physician before the delivery system is used for example to define various deformer lengths. Optionally, the threading on the nuts is flexible enough (or frangible) to allow rod 505 to rotate while the nut is held in place.

Optionally, a pin 528 in nut 524 extends outside of body 502 and serves as a marker on a scale, to show a status and/or degree of deformation. Alternatively, pin 528 may be mounted on nut 508.

Optionally, one or more markings 526 are provided on overtube 528, for example, to indicate its retraction degree and/or to assist in determining a depth in the body.

In an exemplary embodiment of the invention, inadvertent motion of overtube 208 during insertion into the body is prevented using a locking mechanism. FIGS. 5C and 5D show a detailed view of an exemplary locking mechanism in operation, in which motion of a nut allows a lock holding the overtube in place to be released and allows the block to be moved, optionally by the same nut.

In FIG. 5C, a lock 514 locks block 512 to a nub 516 in body 502. A bottom surface 530 of nut 524 prevents lock 514 from moving out of the way, by pressing against an upper surface 532, thereof. This prevents inadvertent motion of block 512 (and overtube 208), for example during insertion.

In FIG. 5D, once nut 524 is retracted sufficiently, lock 514 is not blocked and will disengage from nub 516 when block 512 is retracted by nut 524, for example by an inclined surface 538 of lock 514 sliding past an inclined surface 536 of nub 516.

It should be noted that a same delivery system can be used for devices where deformer 100 stays in the body and devices where deformer 100 is part of system 500.

Optionally, a tensioning state is provided in which after deformer 100 is deployed, additional motion of pusher 206 is provided without motion of overtube 208, for example, to tighten deformer 100. In one example, threading 522 ends in a manner that allows free rotation relative to nut 524, but no axial advance (e.g., a stop), without a corresponding end to threading 506. In another example, all of rod 505 is moved axially, which also may include motion of block 512, if suitably coupled to rod 505.

Optionally, threadings 522 and/or 506 are non-uniform, for example, to provide a certain non-linear relationship between the motions of pusher 206 and overtube 208.

Optionally, system 500 is used to inject cement or another material into the vertebra. In some embodiments of the invention, cement (or any other material, such as bone chips) is injected after deformer 100 is removed or after system 500 is removed. Alternatively, cement is provided through system 500. In an exemplary embodiment of the invention, rod 202 is hollow and cement is provided from a cement source 513 through a tube 511 connected to rod 202. Optionally, rod 202 is apertured at a distal portion underlying deformer 100 (after deformation) and/or at its distal end 204.

In an exemplary embodiment of the invention, the mechanical gain of the system is such that one rotation of the handle causes a 5 or 6 mm shortening of the deformer. Optionally, a gear with varying radius (or other non-linear gear) is used, so that the mechanical gain changes as deformer 100 is deformed. In an exemplary embodiment of the invention, such variations in mechanical gain assist in the application of larger forces at the end of the deformation.

Alternative Delivery Systems

While linear compression of deformer 100 is shown, in other embodiments, rotational motion is provided, for example, by pusher 206 rotating relative to rod 202, optionally being threaded on it.

Optionally, system 500 includes means to limit the forces applied to the vertebra. Optionally, a mechanical fuse is provided in system 500 so that if a threshold force is exceeded, the fuse tears or slips and no further or greater force is/are applied. Optionally, pin 510 serves as such a fuse. Optionally, a string (not shown) is used to retract nut 508 if pin 510 tears. Optionally, pin 510 is designed to bear 20 Kg without tearing. Greater or smaller forces, for example, 10 Kg, 50 Kg or 100 Kg, or smaller or greater forces may be provided as well. In an exemplary embodiment of the invention, the force applied to the deformer is above 1 Kg, 2 Kg or intermediate or greater values.

Optionally, alternatively or additionally, a warning or indication display is used. In one example, a force sensor (not shown) is provided, for example, in rod 202 and which senses the force applied to it by deformer 100. Alternatively or additionally, a strain sensor is provided on rod 202 to measure axial strain. Optionally, such sensors are wired to a warning LED or scale on body 502.

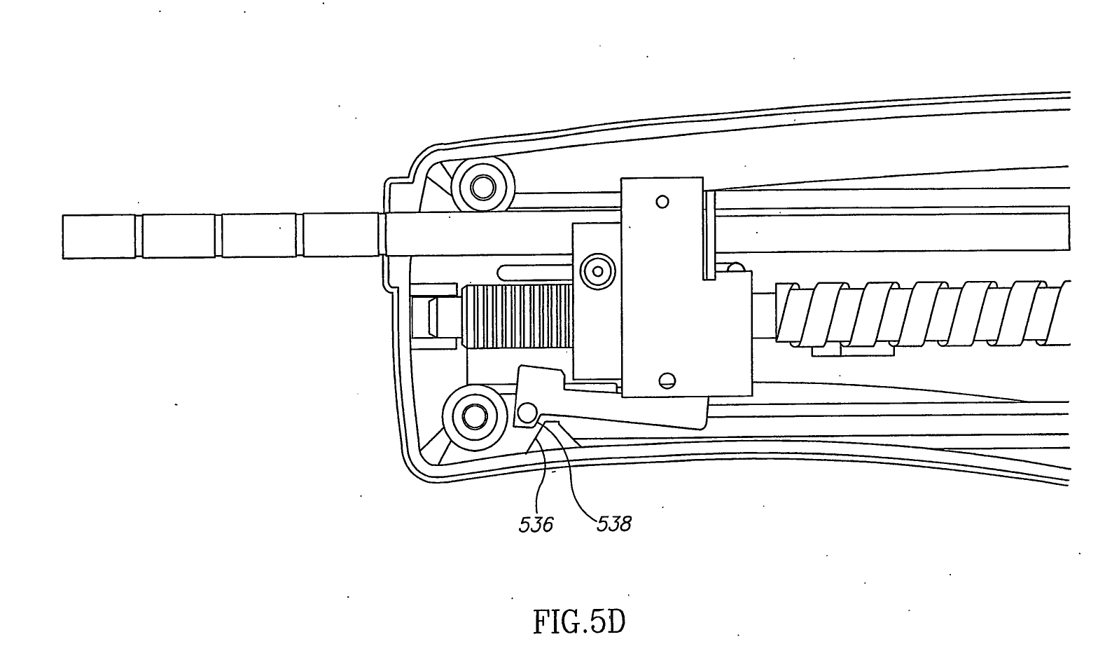

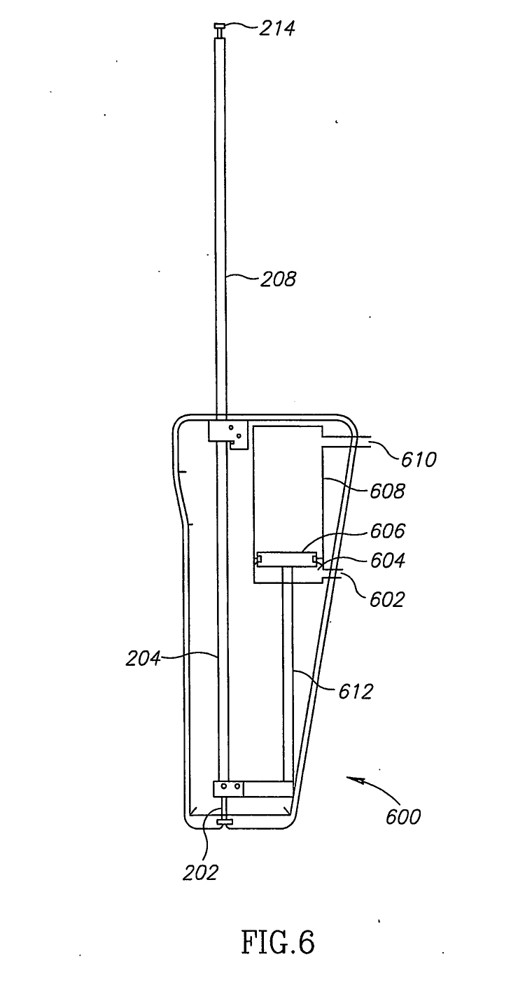

FIG. 6 shows a hydraulic powered delivery system 600, in accordance with an exemplary embodiment of the invention. Fluid (e.g., saline, oil or air) enters a chamber 604 via an inlet 602, pushing against a piston 606 which is free to move in a cylinder 608. Optionally, excess fluid exits through an outlet port 610. Optionally, the hydraulic pressure is manually supplied, for example using a hand pump

Motion of piston 606 is coupled to pusher 206 via a coupler 612. Retraction of overtube 208 is optionally provided using a method as described in FIG. 7A, below.

FIG. 7A is a cross-sectional view of an alternative delivery system 700, in accordance with an exemplary embodiment of the invention. A handle 702 is attached to a body 704 and can be rotated relatively thereto. An optional power gear 706 reduces the motion of rotation, to increase its mechanical gain and turns a belt 708. A block 712 rides on the belt and pushes pusher 206. Optionally, belt 708 slips when it attempts to apply too great (and possibly dangerous) a force.

In an exemplary embodiment of the invention, the following mechanism is used to couple a retraction of overtube 208 with advance of pusher 206. A tongue 714 interconnects a pin 710 of block 712 with a pin 720 of a block 718 coupled to overtube 208. Prior to pin 710 reaching an inclined section 716 of tongue 714, a hook 722 prevents retraction of overtube 208. Once inclined section 716 is reached, tongue 714 moves and pin 720 slides along an inclined surface 724 (shown as dashed). This sliding causes retraction of overtube 208.

Inclined section 716 and surface 724 can also be non-linear.

FIGS. 7B and 7C illustrate a tension based delivery mechanism 780, in accordance with an exemplary embodiment of the invention. Instead of a pusher rod or tube 204 (e.g., as in FIG. 7A), a disc 782 is provided which compresses deformer 100. In an exemplary embodiment of the invention, a wire 784 is attached to disc 782 at a point 788, and travels along a lumen 786 along deformer 100 to distal end 214 thereof Optionally, lumen 786 is formed between rod 202 and deformer 100. Optionally, lumen 786 is formed as a groove in rod 202.

At distal end 214, a curved lumen 790 turns wire 784 back towards disc 782. Optionally, wire 784 exits disc 782 via an aperture 794 therein.

In operation, when wire 784 is pulled back, disc 782 advances and deforms deformer 100.

In an alternative embodiment, disc 782 is advanced by pressure of a fluid, rather than by tension from a wire.

In an alternative embodiment, disc 782 engages rod 202 using a threading on rod 202 and/or disk 782. Advancing of disc 782 is optionally by rotation of disc 782.

It should be noted that the forces applied by deformer 100 may be relative small until the final compression. Thus, even a flexible delivery system is not expected to deformed at least during most of the deformation process. Optionally, the delivery system is provided via an endoscope or is otherwise navigable. Optionally, such flexible delivery is used for delivering some of the implants described below. Optionally, overtube 208 is flexible and is inelastic enough to prevent undesired radial distortion of deformer 100 except where desired.

Optionally, one or more of rod 202, pusher 206 and overtube 208 are bent or bendable, for example, for non-linear endoscope use.

FIGS. 7D and 7E illustrate an alternative deforming mechanism, in which a pliable material is distorted from a narrow diameter to a greater diameter.

In FIG. 7D, a portion 793 of soft material, such as silicon is contained within overtube 208. Optionally, portion 793 comprises a stiffer bag with an inner contents which are softer. A wire 796 optionally interconnects a distal tip 797 with a base section (not shown).

In FIG. 7E, a pusher tube 794 is advanced, forcing the silicon to have a radially extended shape.

In an alternative embodiment of the invention, portion 793 is elastically pre-disposed to be radially extended. Thus, retraction of the overtube allows portion 793 to extend radially, even without advancing pusher tube 794. Pusher tube 794 is optionally retracted in order to radially compress portion 793.

In an exemplary embodiment of the invention, portion 793 is formed of a hard plastic material, such as polyurethane, manufactured by Cardiotech, Inc. with a hardness of Shore 80A and having an elongation of at least 200%, for example 475%. Other values may be used as well, for example, depending on the implant size and its desired compressibility. Thus, portion 793 can withstand considerable forces (e.g., spinal forces) without significantly distorting. In use, a strong over tube is provided, into which portion 793 is pushed, for example, using a piston pushing portion 793 into a funnel terminating at the overtube. Portion 793 is then pushed out of the overtube and into the intra- or inter- vertebral space, to expand. Optionally, portion 793 is mounted on a tube or attached to a wire, so that it can be removed by retracting into the overtube. Optionally, one or more guiding bars are provided on sides of portion 793 during removal, to guide portion 793 into the removal tube.

Optionally, one or more of the above delivery methods, systems and mechanism is used for delivering or deploying a cage device and/or other devices, for example as described in PCT publication WO 00/44319 and WO 00/44321, the disclosures of which are incorporated herein by reference.

FIG. 7F shows an alternative delivery system 2000, in which a flexible pushing element 2002 is used. In an exemplary embodiment of the invention, the use of a flexible pushing tube allows shortening of the deliver system, thereby potentially making it less cumbersome.

A handle (not shown) is attached to a shaft 2004. An optional set of gears 2006 conveys the force to a drive wheel gear 2008.

An inner gear section 2010 of wheel 2008 is used to retract an overtube 2022, as described below.

An outer gear section 2012 engages a band 2014 and, when wheel 2008 rotates, applies force to pushing element 2002, either in a forward or in a backwards direction. Band 2014 may have an interlocking design with section 2012 other than a gear design. Optionally, band 2014 is attached to element 2002 at a connection 2016.

In an exemplary embodiment of the invention, pushing element 2002 is slotted 2018 along at least part of its length, to accommodate a rod 2020 which is optionally provided for holding the distal end of the delivery system (e.g., inside the vertebra). In some embodiments, there is no such forward end which does not move relative to a body 2024 of the delivery system. A guiding rail is optionally provided in body 2024, for riding in slot 2018.

In an exemplary embodiment of the invention, overtube 2022 is retracted by a partial gear section 2026 being engaged by inner gear section 2010. Rotation of section 2010 causes movement of a finger 2028. A projection 2030 that is mounted on a body 2032 coupled to overtube 2022 (see FIG. 7G), is moved by finger 2028, to retract the over tube. The gear ratios (which may also include non-uniform gears) can determine the relative retraction of the overtube and advancing of pushing element 2002. Further, a starting distance between finger 2028 and projection 203 can determine a dead motion during which the overtube does not retract which element 2002 is advanced. Alternatively, pushing element 2002 may have freedom (e.g., band 2014 may be loose, so that the overtube may be retracted without pushing on element 2002).

When retraction is completed, gear section 2026 optionally disengages from gear section 2010. In one example, a small spring may be provided to pull section 2026 away. In another example, the gears of the two sections do not engage once disengaged.

When removing the deformer from the body, a mechanism shown in FIG. 7G, below, prevents advancing motion of overtube 2022, while allowing retraction of pushing element 2002, so that the deformer is undeformed.

In some cases, the undeforming of the deformer may cause forward motion of distal portion of the delivery system. For example, if during retraction the deformer does not completely regain its narrow configuration, a thickened section of the deformer may catch on the opening drilled in the vertebra and further “retraction” of the pushing element (2002) will actually cause an advancing of the distal end, possibly causing damage. In an exemplary embodiment of the invention, this forward motion is prevent by resting the body of the delivery system on the pedicles of the spine. In an exemplary embodiment of the invention, a cannula is used to deliver the deformer and this cannula rests on the pedicle or other bone section. While the depth of penetration of overtube 2022 into the body may not be known exactly a prior, once the delivery is inserted, the depth of penetration is fixed. At this time, a spacer is optionally used to rest body 2024 (or in other delivery systems, even of other types, their body) on the cannula. In an exemplary embodiment of the invention, a resting extension 2034 is selectively advancable using a control 2036, which locks in place using a series of teeth 2038, which optionally act as a ratchet. A potential advantage is that if the surgeon leans on the delivery system, the force is applied to the pedicle, rather than to the weak (or fractured) vertebral side walls.

In an alternative embodiment, the overtube is advanced into and/or remains, in the vertebra during retraction of the deformer. Optionally, one or more bars and/or a mesh are extended from the distal tip of the overtube to guide the deformer back into the overtube. In an exemplary embodiment of the invention, the mechanism of FIG. 7G, below, is modified to merely prevent advancement of the overtube (e.g., the overtube remains in the vertebra), by the ratchet mechanism locking to body 2024 rather than to pushing element 2002.

FIG. 7G shows a locking mechanism which retracts overtube 2002 during retraction of pushing element 2002. A ratchet 2040 is mounted on body 2032, for example via a pin 2042. A tip 2044 of ratchet 2040 is in contact with pushing element 2002. A spring (not shown) optionally urges this contact. Optionally, pushing element 2002 is relative rigid over part of its length, for example being formed of axially attached sections.

When pushing element 2002 moves distally, ratchet 2040 is pushed away. However, when pushing element 2002 is moved proximally (e.g., away from the patient's body), ratchet 2040 locks to element 2002 and overtube 2022 is pulled back with element 2002.

FIG. 7H shows a non-integral spacer 2050 for use with spinal delivery systems. In an exemplary embodiment of the invention, spacer 2050 is formed of two or more parts 2052 and 2054 which can be configured, for example by telescoping, to have a selectable total spacer length. In an exemplary embodiment of the invention, an inner thread (not shown) in part 2052 matches an outer thread on part 2054. Optionally, a slot 2056 is formed in the spacer, so that the spacer can be mounted on a delivery tube after the fact and/or conveniently. Optionally, one or both of two ends 2058 and 2060 of the spacer are adapted for attachment to the relevant tools, for example, to the cannula or to the body of the delivery system.

Variations on Vertebral Treatment

As noted above, in some embodiments of the invention, cement is injected while deformer 100 is inside the body or after it is removed. In some embodiments, the cement or other material is injected into a balloon/mesh, before or after deformer 100 is removed. In some embodiments, no cement or other material is injected.

In a particular variation, a deformer is removed and a metal cage device is inserted. This device may then be filled with materials such as cement or bone chips, as described herein.

A potential advantage of injecting cement after a deformer 100 is in place, is that as most of the volume is filled by the deformer, a smaller amount of cement is needed and there may be less danger of leakage or undesirable migration of the cement. Also, as a void is already created and held open, lower pressures may be used to advance the cement.

Another potential advantage is that the cement can be sweated out through deformer 100, thus possibly holding deformer 100 together and/or assist in providing a uniform distribution of cement. Optionally, pockets are formed for cement between different sets of leaves, which pockets are generally decoupled, so that increased pressure in one pocket does not necessarily cause leakage from another pocket.

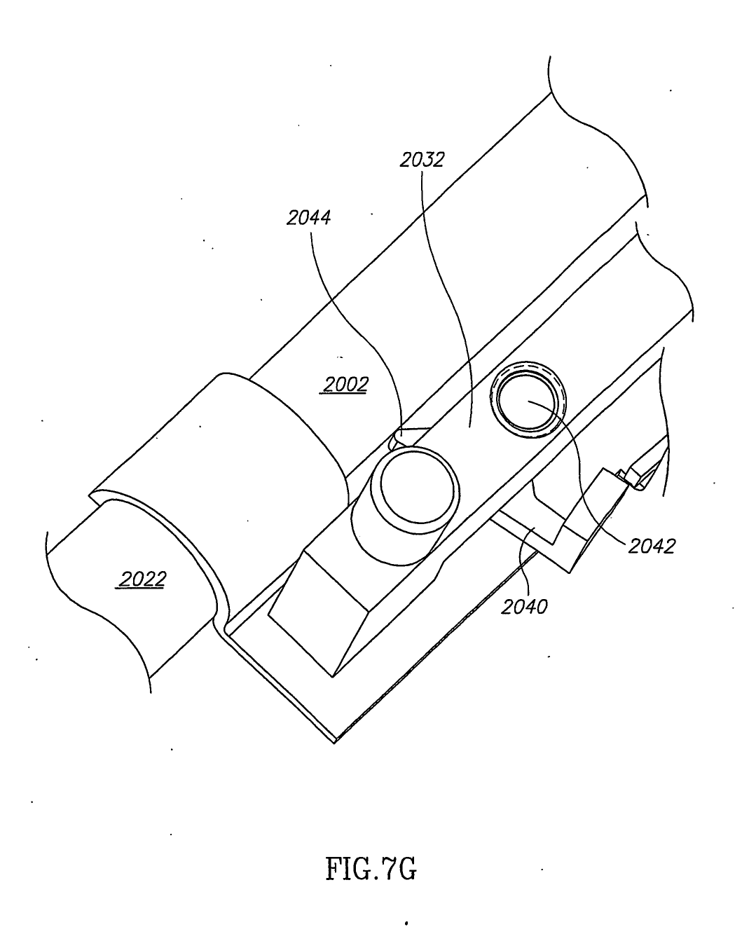

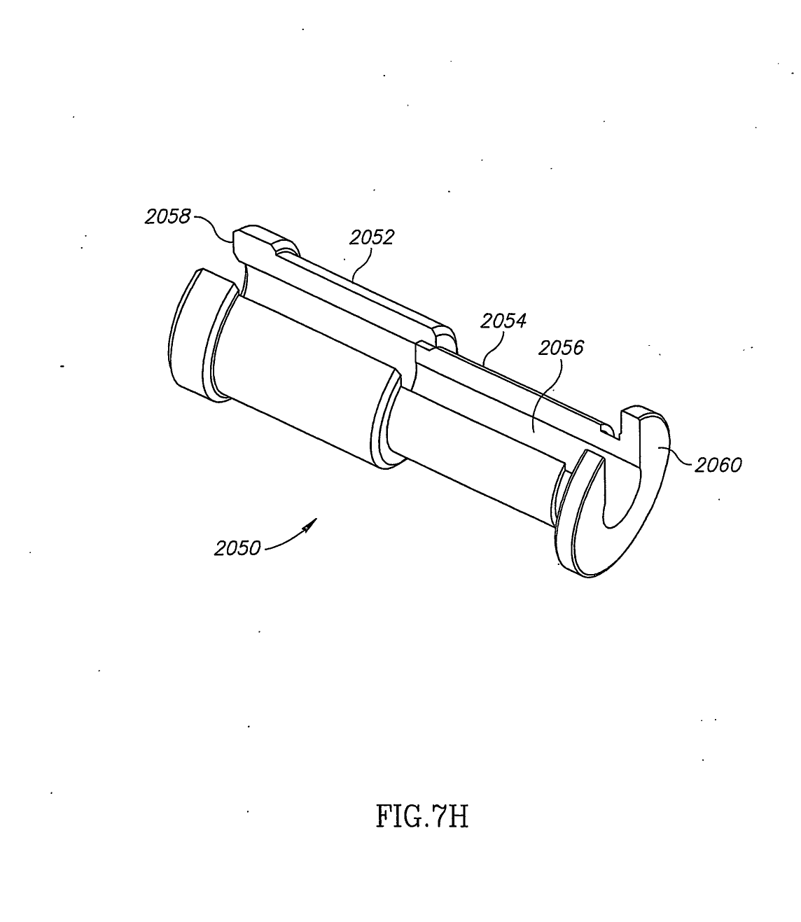

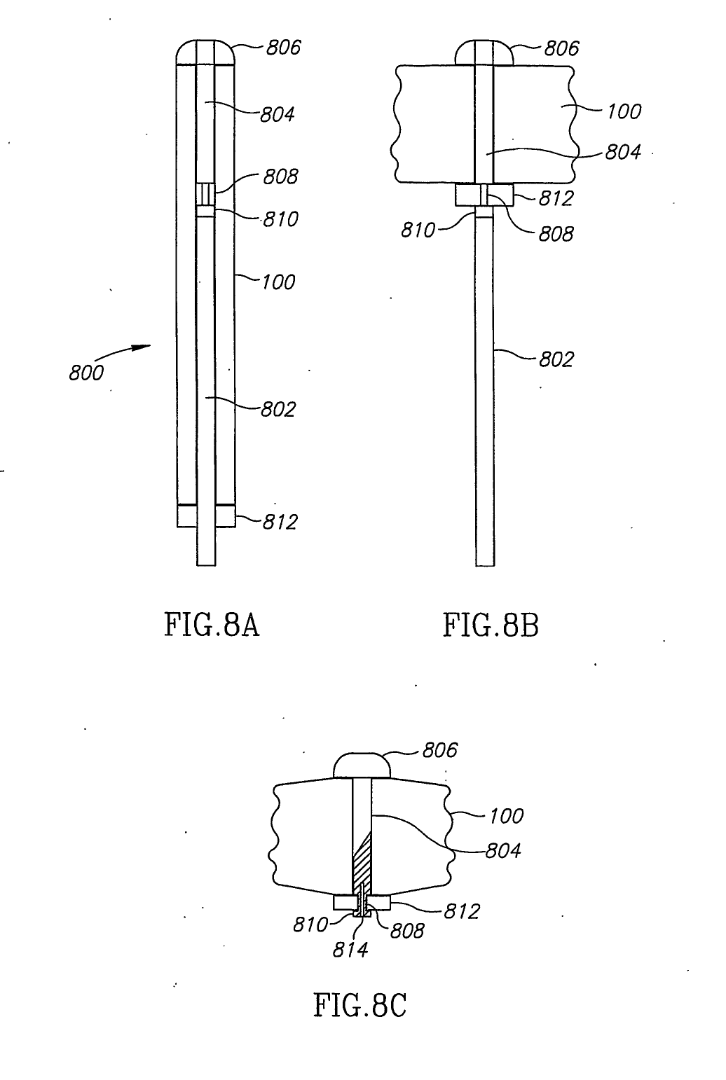

FIGS. 8A-8C illustrate a deformer release mechanism 800 in accordance with an exemplary embodiment of the invention. Deformer 100 is mounted on a rod 802 that engages an extension 804 with a distal tip 806. In FIG. 8A, deformer 100 is undeformed. In FIG. 8B, a disc 812 has been advanced so that it deforms deformer 100 and engages a narrowing 808 in extension 804. For example, disc 812 can be super-elastic. Retraction of disc 812 is optionally prevented by a base 810 of extension 804.

In FIG. 8C, rod 802 is removed from extension 804, for example, by being unscrewed from a recess 814 which engages a tip thereof (not shown, for example using threading). To remove deformer 100 from the body, a disc 812 can be shape memory and a cooling fluid provided to make it pliable and easy to remove.

In an exemplary embodiment of the invention, an implanted deformer is removed using a guide wire that remains attached to it and which is used to guide the deformer into a removal tube. The lips of the removal tube optionally release the axial locking. In one example, the lips bend disc 812 out of base 810. Alternatively, a cutting tool is used which cuts the base and/or the disk.

In an example of an automatic deformer release mechanism, disc 812, when entering recess 812, shears one or more wires (not shown) which pass in recess 808 and attach extension 804 to rod 802.

Optionally, in some embodiments of the invention deformer 100 is formed of Titanium or another metal, for example as described in earlier applications of the assignee of the present application.

Variations on Deformer Design

A deformer may have various cross-sectional shapes, for example, being a rectangle, a square, a circle, an ellipse and/or a concave shape. Optionally, the crosssectional shape and/or dimensions change along the axis of the deformer. Optionally, various shapes are achieved by suitable cutting of slot lengths. Alternatively or additionally, a deformer shape may be achieved by cutting the deformer after it is deformed to have the desired geometry and then un-deforming the deformer. In some embodiments, a deformer of a predetermined geometry is used. Alternatively or additionally, the deformer is cut or selected based on 3D or 2D images of the vertebra to be treated. The cross-section may be, for example rotationally symmetric, mirror symmetric or asymmetric.

The axial shape may also be of various types, for example, uniform, lordotic (at one side a greater diameter than the other), ellipsoid (narrower diameter at both ends), hour-glass, or varying (e.g., diameter increases and decreases several times along its length).

The material from which the deformer is made can be, for example, of uniform thickness and uniform properties. Alternatively one or both of the thickness and material properties can vary, resulting, for example, in a deformer which has a non-uniform deformation. The varying can be, for example, in an axial and/or angular directions.

Optionally, properties of a deformer are made to vary by varying one or more of length, direction, width, linearity, and/or spatial density of slots. Alternatively or additionally, alignments of pairs of slots (which define a leaf) is varied. For example, slots can have a helical pattern, be arranged in lines and/or vary in axial and/or radial densities.

Optionally, in some embodiments of the invention, no slots are provided. For example, the deformer may be twisted or compressed (and is optionally made more elastic). Alternatively or additionally, the slots do not reach through the thickness of the deformer, for example being only inside and/or only outside.

In an exemplary embodiment of the invention, one side of the deformer is made softer and/or thinner so that deformation will be preferable in a certain direction.

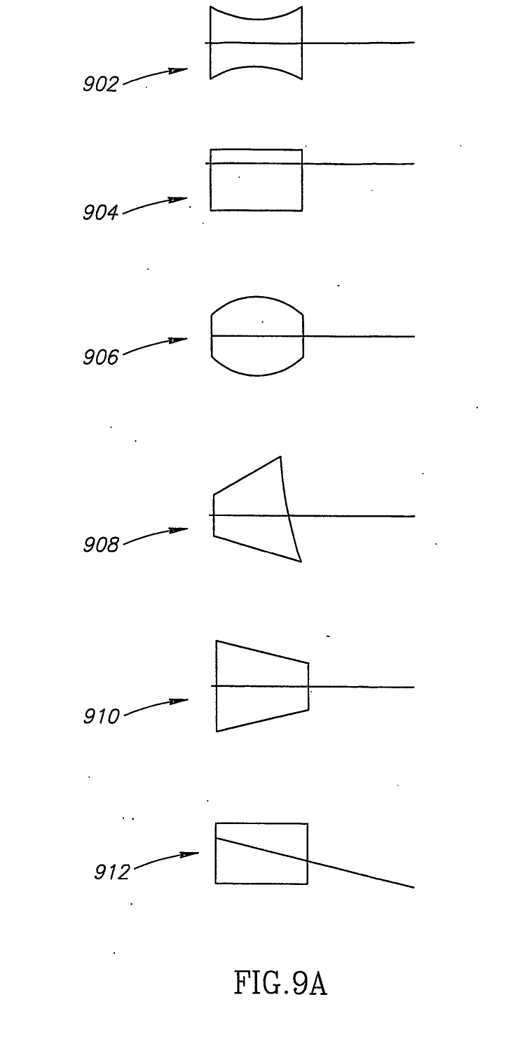

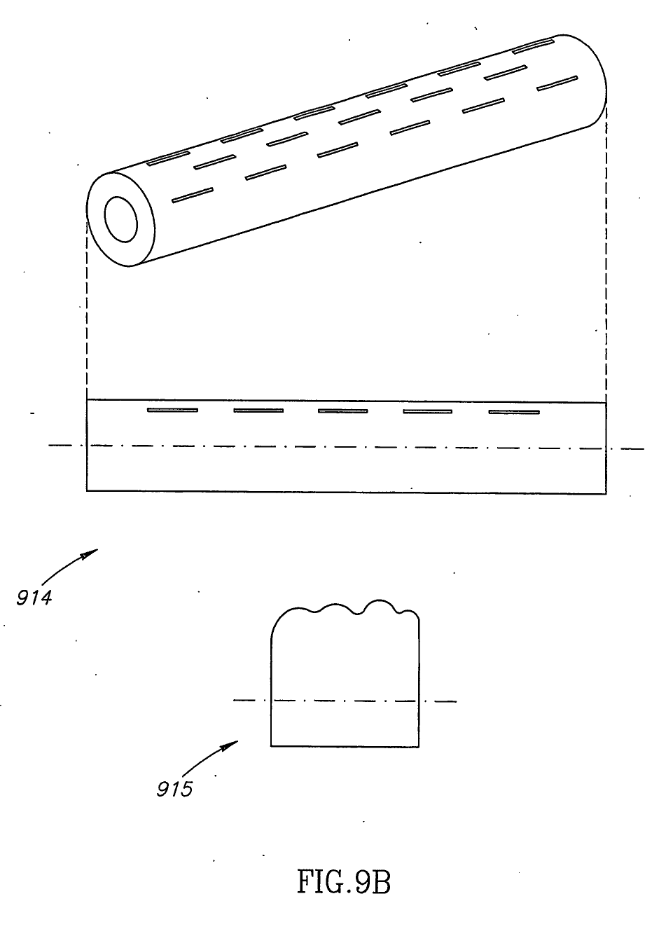

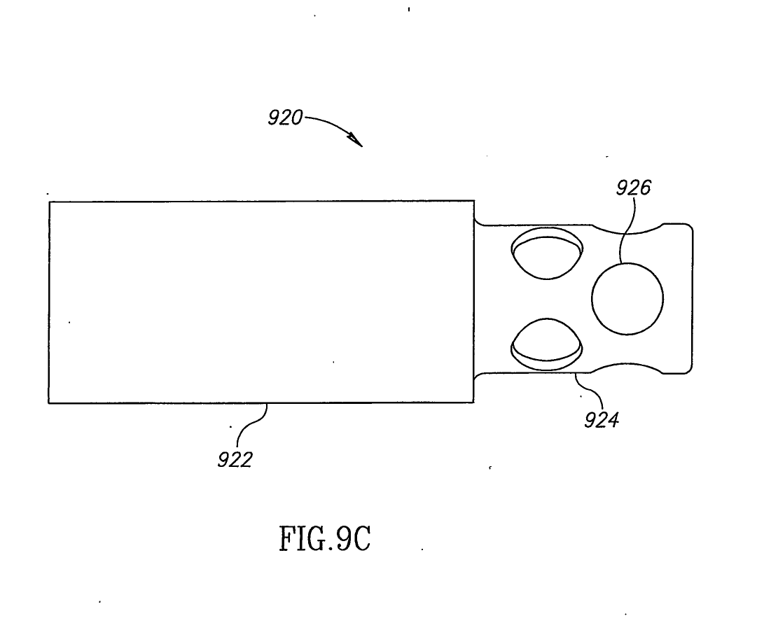

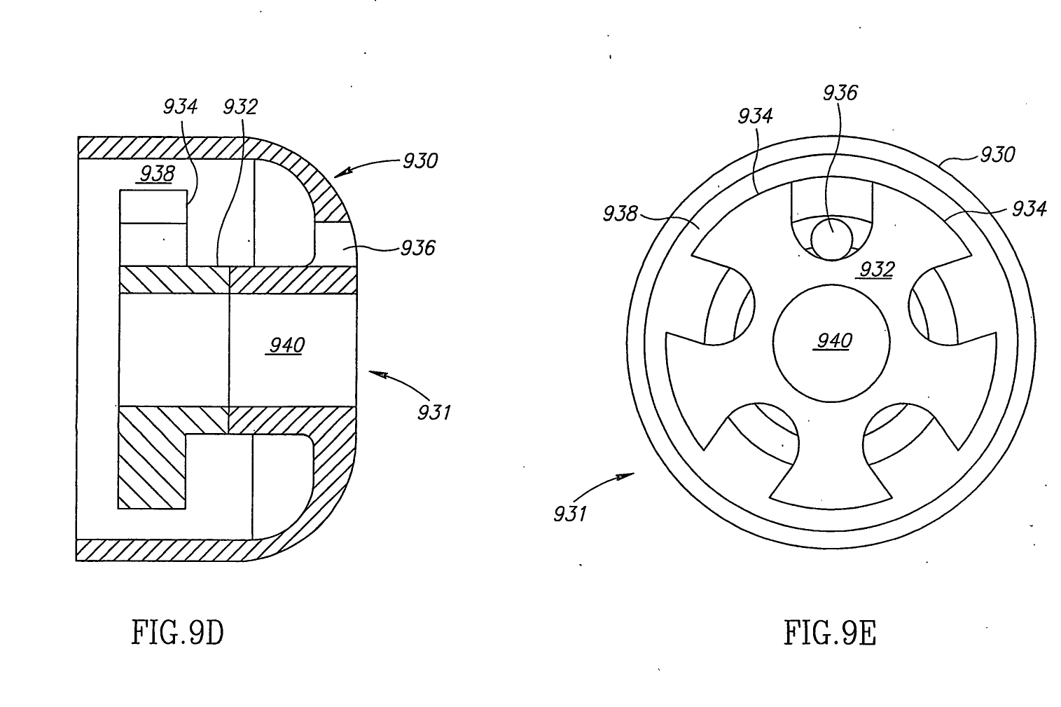

FIG. 9A shows six different exemplary axial profiles, hourglass (902), off-axis symmetric (904), ellipsoid (906), lordotic (908), inverse lordotic (910), and off-axis asymmetric (912). Other off-axis designs can be used.

In a particular example, FIG. 9B shows a deformer 914, in which slots are formed on one side, resulting in deformation (915, side view) which is not symmetric with respect to the axis.

The contact area between the deformer and the tissue, can be, for example, bumpy (as shown in FIG. 1), or smooth, for example, if the device is encased in a bag, or if the ends of the leaves are treated so that they are softer than other parts of the deformer.

In many of the embodiments described, an inner rod is used to lock the deformer. Optionally, a wire is used to lock the deformer by interconnecting the two ends of the deformer. Optionally, the ends fold in, at least slightly, so that they are protected from outside tissue, for example, so they do not contact bone.

Optionally, the inner bar is made of a super elastic or shape memory material that determines the final shape of the deformer, once released and/or during release. For example, the inner rod can be pre-trained to achieve a curved or spiral shape, in 2D or in 3D.

Optionally, the deformer is shaped so that multiple deformers will interlock or fit side by side, for example, a concave depression in one deformer matching another deformer. Optionally, when required, multiple deformers are implanted and/or deformed at a same time or even simultaneously.

Optionally, one or more leaves have defined thereon hairs or projections to engage tissue and/or encourage ingrowth or adhesion.

One property of deformers in accordance with some embodiments of the invention is that leaves are supported by leaves. As a result leaves at the ends lack some support. Optionally, the end leaves are made shorter. Alternatively or additionally, these end leaves are made stiffer. Alternatively or additionally, these end leaves bend over the axis. Alternatively or additionally, a greater number of leaves and/or axial or radial leaf density are provided at the end.

FIG. 9C shows a proximal end 920 for attaching a deformer thereto, in accordance with an exemplary embodiment of the invention. End 920 comprises a tubular section 922 adapted to engage a pusher 906, for example by contact, adhesive or threading. End 920 comprises an aperture section 924 including a plurality of apertures 924, on which proximal end 116 of deformer 100 can be mounted and melted on to ensure engagement.

FIGS. 9D and 9E illustrate a distal end cap 931 for attaching a deformer thereto, in accordance with an exemplary embodiment of the invention.

An outer body 930 and an inner rosette 932 having a plurality of petals 934 define between them a lumen 938 into which distal end 114 of deformer 100 is inserted. Heat is then applied to melt the plastic into the metal. Alternatively or additionally, adhesive may be used. Optionally, an aperture 936 is provided for venting air, if required. Optionally, a lumen 940 is defined through rosette 932 and body 930, and is optionally threaded to engage an end 214 of rod 202.

The length of a deformer (in a deformed state can vary depending on the application, for example, being between 2 mm and 100 mm, for example, 10 mm, 20 mm, 30 mm, 40 mm, or any smaller, larger or intermediate values. A ratio of axial shortening, can be, for example, 1:2, 1:3, 1:4, 1:5, 1:8, 1:10, or any smaller, intermediate or greater ratio. A ratio of radial increase, can be, for example, 1:2, 1:3, 1:4, 1:5, or any smaller, intermediate or greater ratio. The radius of an undeformed deformer can be, for example, 1 mm, 2 mm, 3 mm, 4 mm, 5 mm, 7 mm, 10 mm or any smaller, intermediate or greater radius. The thickness of the deformer material can be, for example, 0.5 mm, 1 mm, 2 mm, 3 mm, or any smaller, intermediate or greater thickness. Axial and/or radial leaf density per length unit can be, for example, 1:4 mm, 1:3 mm, 1:2 mm 1:1 mm 1:0.5 mm or any smaller, intermediate or greater per unit density. Leaf length can be, for example, 1 mm, 3 mm, 5 mm, 7 mm, 10 mm, 20 mm, or any smaller, intermediate or greater length. The degree of deformation may depend, for example on the desired tradeoffs, size of access hole, existing holes in bone and/or other medical conditions and/or mechanical properties of the material and/or design being used.

Leaf designs and patterns may be patterned upon those shown in PCT publication WO 00/44319, in accordance with some embodiments of the invention.

Materials

In an exemplary embodiment of the invention, the material used has a shore hardness between 50A and 90D, for example, between 90A and 60D. Intermediate hardness values, may be provided as well, for example, at any one of ten equidistant intermediate hardness values. Optionally, the material is selected so that individual leaves can be moved by the forces applied by the bone (e.g., a spine), while multiple leaves will be able to support each other against such forces.

In one example, a device as described above, with cylindrical diameter, deforms from a diameter of 5 mm to 15 mm, is 20 mm long, made from polyurethane, has a shore hardness 90A, is form of a tube 1.6 mm thick and was bench tested to lift 60 Kg. Other thicknesses, such as 0.5 mm, 1 mm, 2 mm and smaller, intermediate or greater thicknesses, maybe used.

In an exemplary embodiment of the invention, pliability and/or leaf density are selected to control a desired space filling effect.

The material used is optionally elastic.

Optionally, a compressible material is used. In one example, a fabric, for example Gore-Tex, Dacron or a metal mesh are used.

Optionally, a natural material, such as cotton or collagen are used.

Optionally, a tradeoff between material pliability and conformance of the device is achieved. For example, as the material is made softer, the device may be more conforming during implantation/use and possibly even after final tightening of the device.

Optionally, deformer 100 is coated with various materials, for example, an adhesive, osteo-conductive materials, growth promoters, anti-inflammatory, antibiotics, radioactive materials or other a materials known in the art.

Optionally, deformer 100 is made smooth to assist in its removal after a time.

Optionally, deformer 100 is made degradable so that it degrade after time and do not need to be removed and/or is made partially degradable so that tissue igrowth can occur. Optionally, different parts degrade at different rates, for example, the inner locking bar degrading only after a long time or not at all.

In an exemplary embodiment of the invention, deformer 100 is formed of a composite material. In one example, deformer 100 is manufactured by stringing beads of various materials and then melting them together to form a tube. Alternatively, a deformer is made from segments which are merely strung together and possibly adhered to each other. Optionally, different beads have different mechanical and/or degrading properties.

FIG. 9F shows another type of composite device, 950, in which a plurality of wires, for example, Kevlar or metal wires 952 are embedded therein. Optionally, the wires are not embedded but are found in channels 954, optionally, allowing relative motion of the wires and deformer 950.

Alternatively or additionally, radially-directed wires may be embedded. Alternatively or additionally, a helical wire is provided. Optionally, one or more radial wires are provided at one or both ends of the deformer. Optionally, such end wires prevent tearing of deformer 950 and may optionally be locked together by a locking wire.

A potential advantage of using wires is to prevent tearing of the deformer. Another potential advantage of using wires is that a deformer may be removed by pulling on the wires, rather than on the deformer. Another potential advantage of using wires is changing material properties locally and/or providing strength where needed.

Artificial Disc Application

In an exemplary embodiment of the invention, deformer 100 is used as an implant for a disc. In an exemplary embodiment of the invention, some or all of the disc material is removed. Alternatively, no disc material is removed. A deformer 100 is then implanted inside the inter-vertebral space, to support, expand and/or replace an existing disc or disc nucleus. Optionally, an implant which curves or curls is used. Alternatively, two implants may be inserted side by side.

Artificial Joint

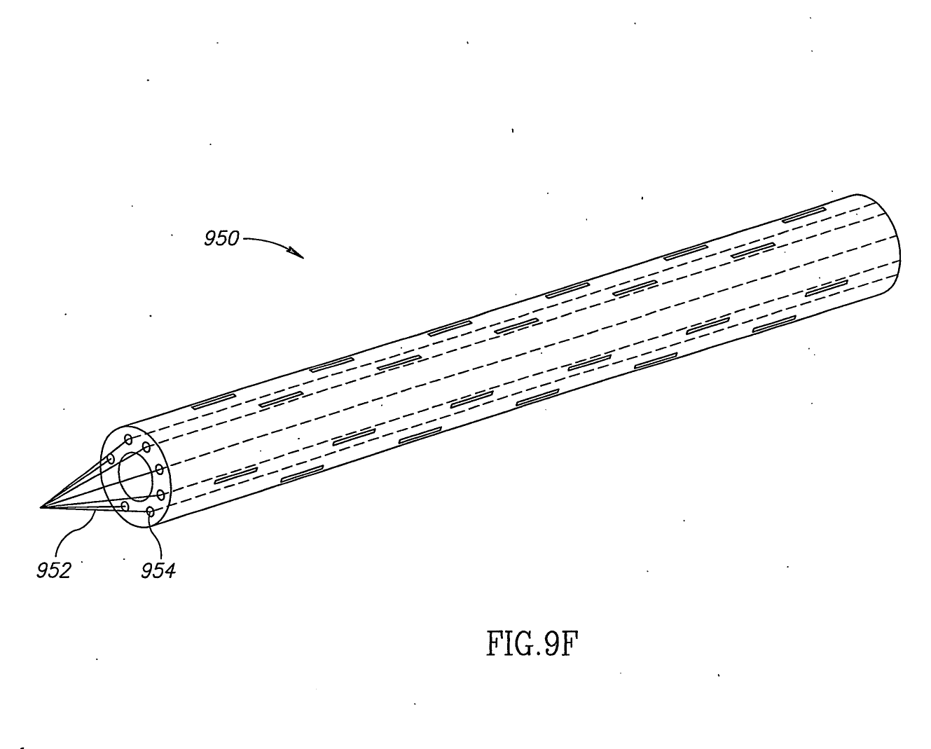

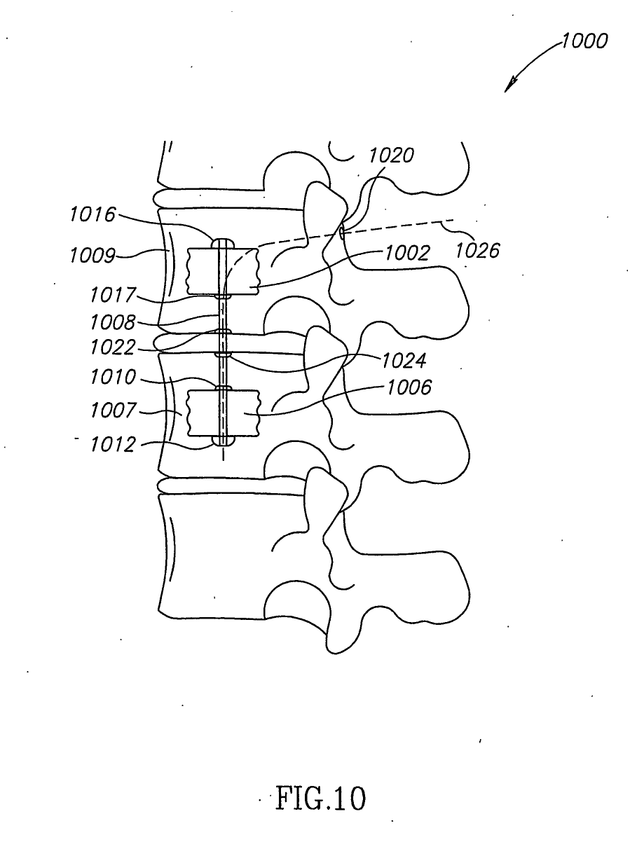

FIG. 10 shows a vertebral joint 1000, in accordance with an exemplary embodiment of the invention. Joint 1000 comprises a first bone-engaging element 1002 engaging an inner volume of a vertebra 1009 and a second bone-engaging element 1006 engaging an inner volume of a vertebra 1007. The two elements 1002 and 1006 are interconnected by a bar or rod 1008, which either includes a hinge or serves as a living hinge between the vertebra. For example, in order to serve as a hinge, bar 1008, can be flat so as to have a preferred bending direction. Alternatively or additionally, bar 1008 is notched on one side, so as to prefer bending in one direction rather than bending back.

In an exemplary embodiment of the invention, device joint 1000 is deployed in the following manner. A cannula is guided to vertebra 1009 and an aperture 1020 made therein. A dotted line 1026 shows a path of a flexible drill (not shown) that enters (and optionally forms) aperture 1020 and then forms an aperture 1022 in the base of vertebra 1009 and an aperture 1024 in a top of vertebra 1007. A guide tube is provided along this path. Joint 1000, in a narrowed diameter state is inserted along this path. A mechanism as described above is used to push a ring or slotted ring 1010 to axially compress engaging element 1006 against a base 1012, while retracting the guide tube. When the compression is completed, ring 1010 can lock. Then, the guide tube is further retracted and compression of element 1002, by a ring 1016 against a base 1014, proceeds. In an exemplary embodiment of the invention, advancing of ring 1010 (for compressing element 1006) is done relative to ring 1014. When completed, ring 1016 can lock. Optionally, a flexible delivery system is used. Alternatively, a hinge is provided at ring 1016.

In an alternatively embodiment of the invention, joint 1000 is used as a replacement finger joint. In this embodiment a substantially straight and rigid delivery system can be used.

Different deformer designs and/or types (e.g., self deforming and actively deformed) may be used for either end of the joint, for example to assist in delivery and/or to match the type of tissue in which the end anchors.

Bone Implants

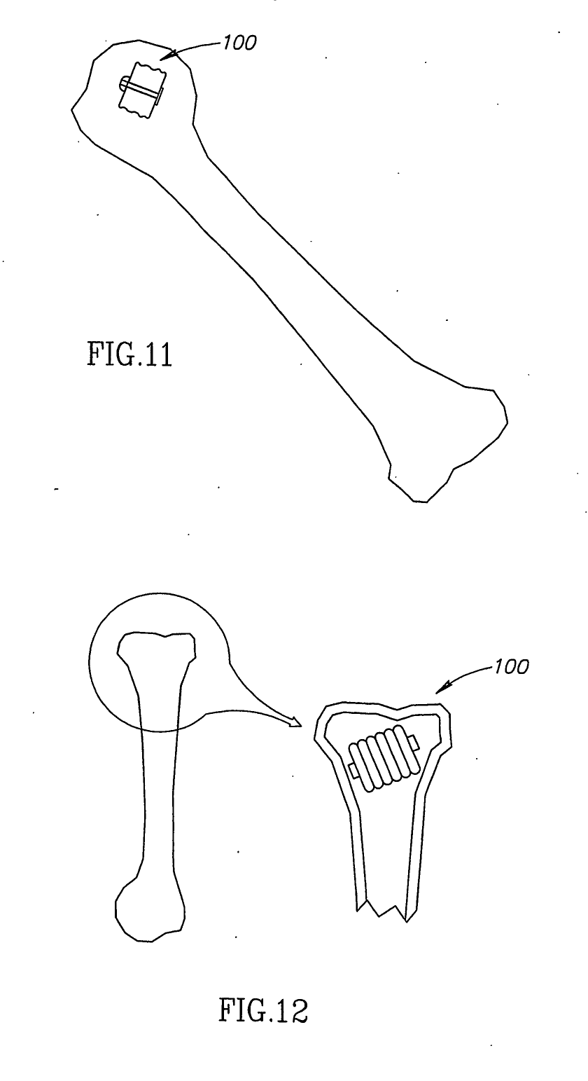

FIGS. 11 and 12 shows the use of an implanted deformer 100 for supporting a humeral head (FIG. 11) and a tibial plateau (FIG. 12), in accordance with an exemplary embodiment of the invention.

In these embodiments, a deformer is inserted near the inside of an end plate of a long bone to support and/or displace a broken and/or sunken bone section. Optionally, the deformer is used on an old fracture, to reset the bone. Optionally, the deformer selected is positioned to press against two cortical bone sections, one stronger and one to be moved or supported. Alternatively or additionally, one side of the deformer may rest on compressed spongy bone. Alternatively or additionally, the deformer is configured to expand radially more in one direction than in another. Alternatively or additionally, the deformer is configured to be wider in one direction than another, so as to preferentially support motion of the deformer in the narrower direction.

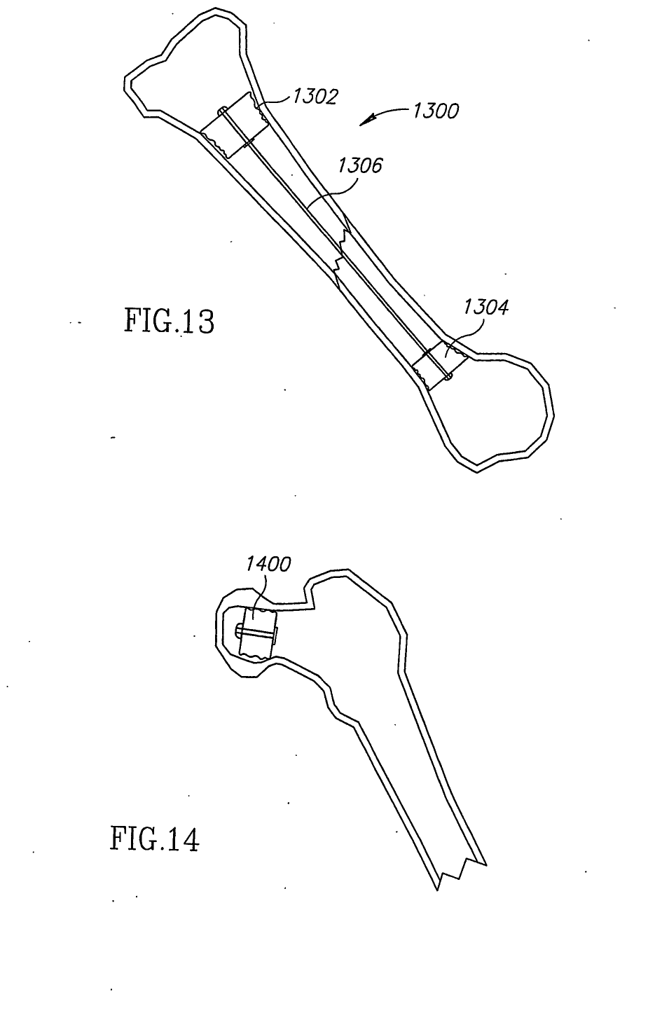

FIG. 13 shows an intra-medullar nail 1300, in accordance with an exemplary embodiment of the invention. Nail 1300 includes deforming elements 1302 and 1304 at either end and a bar 1306 interconnecting them. Optionally, elements 1302 and 1304 degrade in the body so that only bar 1306 needs to be removed. Optionally, bar 1306 also degrade, but at a lower rate. A potential benefit of this design is that the medullar canal may remain mostly undamaged and/or free.

In an alternative nail design, elements 1302 and 1304 are formed of a continuous tube, slit only at the areas of the elements. Optionally, an over tube (not shown) sheaths this single tube between elements 1302 and 1304.

FIG. 14 shows a deformer 1400 in a femoral head application, for example for holding the bone together or for supporting a nail. Alternatively or additionally, a deformer may be used to hold a prosthesis, for example being placed in the femoral medullar channel to hold a hip implant. Optionally, one or more deformers are placed between a cortical bone and an implant, to apply compressive forces to one or both of the bone and the implant.

In a pedicle screw application, or in other applications, a deformer section may be used to apply force and anchor against spongy bone, in addition to or alternatively to hard cortical bone. Optionally, devices such as described in PCT applications PCT/IL00/00458; PCT/IL00/00058; PCT/IL00/00056; PCT/IL00/00055; PCT/IL00/00471; PCT/IL02/00077; PCT/IL03/00052; and PCT/IL2004/000508, the disclosures of which are incorporated herein by reference, may be used with a deformer rather than another type of expandable section.

Dental Implant

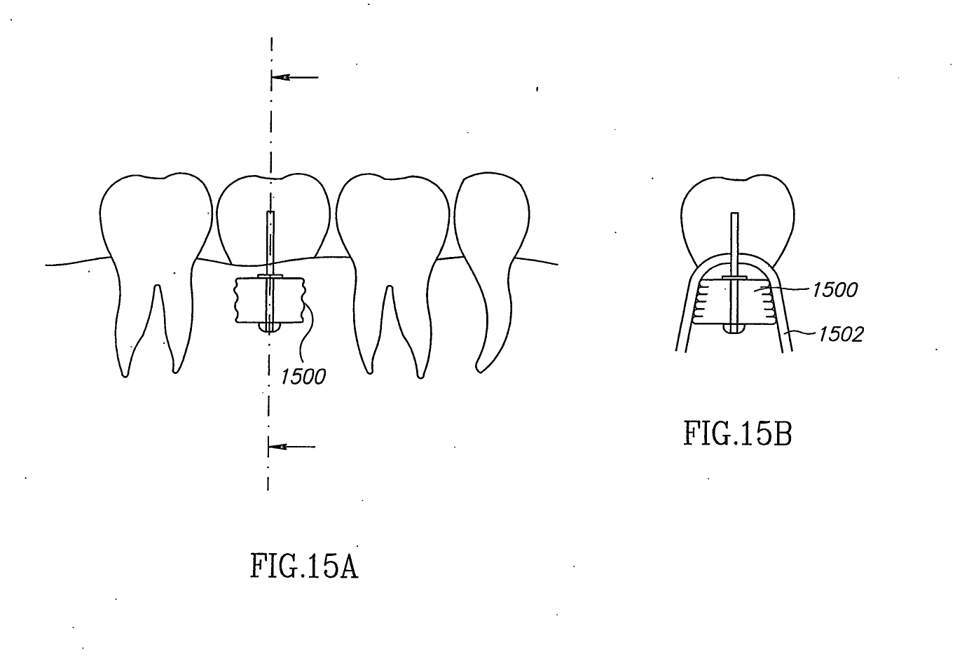

FIGS. 15A and 15B show a dental implant 1500 in accordance with an exemplary embodiment of the invention. One potential advantage of using a deformer-type implant is that in some embodiments an implant can be partially tightened, readjusted and then tightened some more. Optionally, once the implant is completely tightened, a cap is mounted on the implant.

Another potential advantage for dental usage is that a deformer may be able to better grip (and not apply too much force) against a weakened jaw bone (cortical and/or spongy bone). Possibly, the softer materials used in the implant prevent or slow down degradation by wear of the bone, as there is less of a point pressure applied.

Balloon Device

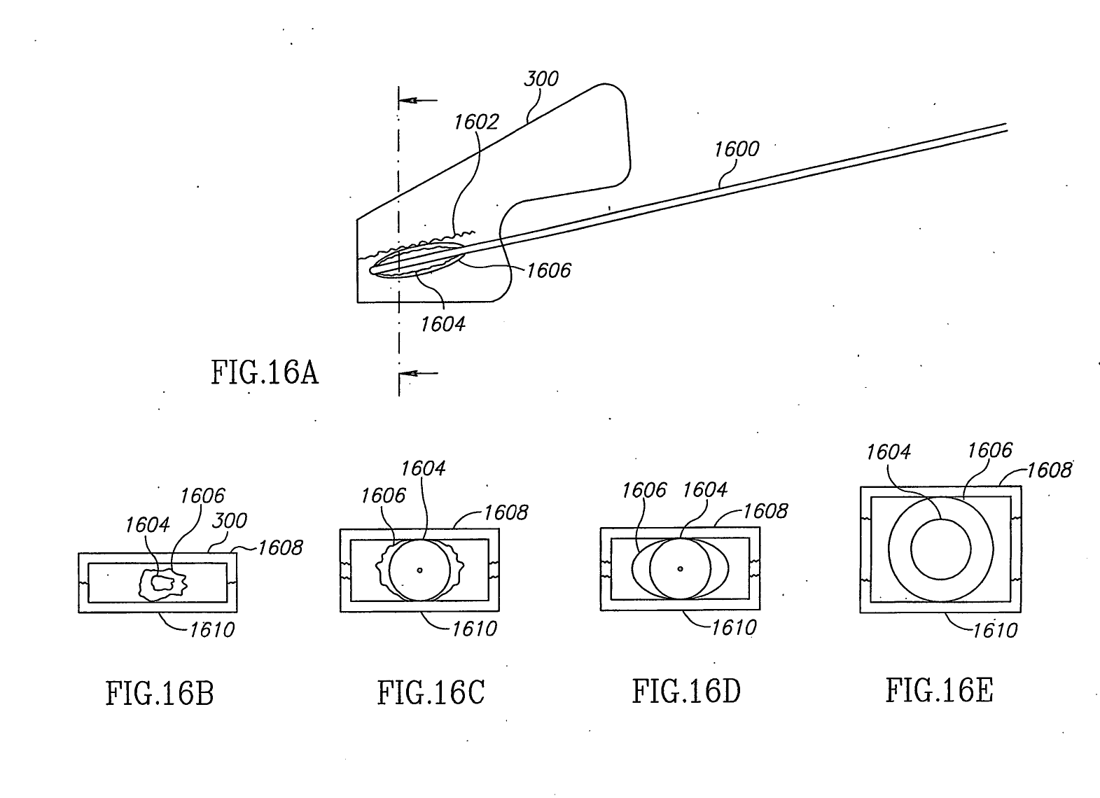

FIGS. 16A-16E illustrate a balloon in balloon expansion device, in accordance with an exemplary embodiment of the invention.

FIG. 16A shows a balloon device 1600 inserted in a vertebra 300 with a fracture 1602. Device 1600 comprises an inner balloon 1604 and an outer balloon 1606.

FIGS. 16B-16E are cross-sectional views along a line II-II, showing the operation of device 1600.

In FIG. 16B, device 1600 is not inflated.

In FIG. 16C, inner balloon 1604 is inflated, partially moving apart end plates 1608 and 1610 and/or fixing balloon 1606 in place.

In FIG. 16D, outer balloon 1606 is partially inflated. As can be seen, balloon 1606 is limited in its ability to reach the sides of vertebra 300, where it might cause damage.

In FIG. 16E, balloon 1606 is further inflated, further spacing apart vertebra end plates 1608 and 1610.

Optionally, inner balloon 1604 is replaced by a deformer, for example of the type described above. Optionally, one or both balloons are biodegradable.

It will be appreciated that the above described methods of implanting and treating may be varied in many ways, including, changing the order of steps, which steps are performed more often and which less often, the arrangement of elements, the type and magnitude of forces applied and/or the particular shapes used. In particular, various tradeoffs may be desirable, for example, between applied forces, degree of resistance and forces that can be withstood. Further, the location of various elements may be switched, without exceeding the sprit of the disclosure, for example, the location of the power source. In addition, a multiplicity of various features, both of method and of devices have been described. It should be appreciated that different features may be combined in different ways. In particular, not all the features shown above in a particular embodiment are necessary in every similar exemplary embodiment of the invention. Further, combinations of the above features are also considered to be within the scope of some exemplary embodiments of the invention. In addition, some of the features of the invention described herein may be adapted for use with prior art devices, in accordance with other exemplary embodiments of the invention. The particular geometric forms used to illustrate the invention should not be considered limiting the invention in its broadest aspect to only those forms, for example, where a cylindrical tube electrode is shown, in other embodiments an rectangular tube maybe used. Although some limitations are described only as method or apparatus limitations, the scope of the invention also includes apparatus programmed and/or designed to carry out the methods.

Also within the scope of the invention are surgical kits which include sets of medical devices suitable for implanting a device or material and such a device. Section headers are provided only to assist in navigating the application and should not be construed as necessarily limiting the contents described in a certain section, to that section. Measurements are provided to serve only as exemplary measurements for particular cases, the exact measurements applied will vary depending on the application. When used in the following claims, the terms “comprises”, “comprising”, “includes”, “including” or the like means “including but not limited to”.

It will be appreciated by a person skilled in the art that the present invention is not limited by what has thus far been described. Rather, the scope of the present invention is limited only by the following claims.

1. A medical grade deformer, comprising:
an axial member; and
a pliable tube mounted on said axial member and adapted to be deformed from a first, narrower diameter, configuration to a second, greater diameter, configuration.

2. A deformer according to claim 1, wherein said tube is slotted through its thickness.

3. A deformer according to claim 1, wherein said tube is not slotted.

4. A deformer according to claim 1, comprising at least one end engaging one end of said tube and adapted to apply compressive force to said tube for achieving said deformation.

5. A deformer according to claim 4, comprising at least a second end one end engaging a second end of said tube and adapted to cooperate with said first end to compress said tube.

6. A deformer according to claim 5, wherein said two engaging ends and said axial member lock to maintain said pliable tube in a greater diameter configuration.

7. A deformer according to claim 1, wherein said tube changes configuration by axial compression thereof.

8. A deformer according to claim 1, wherein said axial member is rigid.

9. A deformer according to claim 1, wherein said axial member is flexible.

10. A deformer according to claim 1, wherein said axial member extends out of said tube and is attached to a handle.

11. A deformer according to claim 1, wherein said axial member comprises a release mechanism for release of said deformer from a delivery system.

12. A deformer according to claim 11, wherein said axial member comprises a locking mechanism for locking of said deformer in a greater diameter configuration in conjunction with release.

13. A deformer according to claim 1, wherein said deformer includes a channel adapted for bone filler flow.

14. A deformer according to claim 13, wherein said channel is formed in said axial member.

15. A deformer according to claim 13, wherein said channel is formed between said axial member and said tube.

16. A deformer according to claim 1, wherein said axial member extends from said tube and is adapted to function as a hinge of a joint.

17. A deformer according to claim 1, wherein said deformer forms a bone attachment unit for a prosthesis.

18. A deformer according to claim 1, comprising an enclosing bag, which surrounds said tube in said second configuration.

19. A deformer according to claim 18, wherein said bag is bio-degradable in the body.

20. A deformer according to claim 18, wherein said bag is porous.

21. A deformer according to claim 1, wherein said deformer defines a general volume in the shape of a cylinder when in said second configuration.

22. A deformer according to claim 1, wherein said deformer defines a general volume in the shape of a truncated pyramid when in said second configuration.

23. A deformer according to claim 1, wherein said deformer defines an axially rotationally asymmetric general volume when in said second configuration.

24. A deformer according to claim 1, wherein said deformer defines a predetermined general volume when in said second configuration.

25. A deformer according to claim 1, wherein said deformer comprises a set of axially contiguous zones with different material properties.

26. A deformer according to claim 1, wherein said deformer has a non-smooth outer surface in said second configuration.

27. A deformer according to claim 1, wherein said deformer is stiff enough, when in said second configuration to resist a trans-axial force of at least 50 Kg.

28. A deformer according to claim 1, wherein said deformer, when in said second configuration has an axial applied force of at least 2 Kg.

29. A deformer according to claim 1, wherein said pliable material has a shore hardness of between 50A and 90D.

30. A deformer according to claim 1, wherein said pliable material is non-metallic.

31. A deformer according to claim 1, wherein said pliable material is polymeric.

32. A deformer according to claim 1, wherein said deformer includes at least one axial thread.

33. A deformer according to claim 1, wherein said deformer includes at least one circumferential thread.

34. A deformer according to claim 1, wherein said deformer, in said second configuration, defines a general volume and wherein said deformer fills at least 30% of said volume.

35. A deformer according to claim 1, wherein said deformer, in said second configuration, defines a general volume and wherein said deformer fills at least 50% of said volume.

36. A deformer according to claim 1, wherein said tube defines a plurality of slots, such that when deformed to the second configuration, a plurality of axially displaced leaves extend from said tube to define said second configuration.

37. A deformer according to claim 36, wherein said tube defines at least three axially displaced leaves.

38. A deformer according to claim 36, wherein adjacent leaves support each other, in said second configurations.

39. A deformer according to claim 36, wherein an end leaf is shorter than a non-end leaf.

40. A deformer according to claim 36, wherein an end leaf is supported, on one side thereof, by an end cap of said deformer.

41. A deformer according to claim 36, wherein adjacent leaves deform each other.

42. A deformer according to claim 36, wherein at least 50% of the leaves are deformed from a plane.

43. A deformer, comprising a non-inflatable substantially non-absorbent deformable non-metallic body having two configurations, a first configuration in which said body has a narrower diameter and a second configuration in which said narrower diameter is greater, wherein said deformer is adapted to remain substantially undeformed under a force of over 10 Kg and wherein said deformer is sized for positioning inside a human vertebra.

44. A deformer according to claim 43, wherein said deformer is adapted to remain substantially undeformed when in a human lumbar vertebra in standing condition.

45. A deformer according to claim 43, wherein said deformer is self-expanding.

46. A deformer according to claim 43, as part of kit including a spinal access tool.

47. A method of spinal surgery, comprising:
inserting a non-inflatable non-absorbent deformable deformer into a vertebra; and
deforming said deformer such that cortical bone of vertebral faces of said vertebra, move relative to each other.

48. A method of treating a bone, comprising:
inserting a unsealed pliable element into the bone; and
mechanically deforming the pliable element such that said pliable element applies deforming force on the bone.

49. A method according to claim 48, wherein said pliable element comprises at least one open aperture of cross-section greater than 0.5×0.5 mm.

50. A method according to claim 48, wherein said bone comprises a vertebral bone.

51. A method according to claim 48, wherein said bone comprises a long bone.

52-61. (canceled)
